(19) United States
(12) Patent Application Publication
(10) Pub. No.: US 2020/0056192 A1
(43) Pub. Date: Feb. 20, 2020

(54) DETECTION OF NUCLEASE EDITED SEQUENCES IN AUTOMATED MODULES AND INSTRUMENTS VIA BULK CELL CULTURE

(71) Applicant: Inscripta, Inc., Boulder, CO (US)

(72) Inventors: Eileen Spindler, Boulder, CO (US); Amy Hiddessen, Boulder, CO (US); Phillip Belgrader, Pleasanton, CA (US); Charles Johnson, Boulder, CO (US); Clint Davis, Boulder, CO (US)

(21) Appl. No.: 16/540,767

(22) Filed: Aug. 14, 2019

Related U.S. Application Data
(63) Continuation-in-part of application No. 16/399,988, filed on Apr. 30, 2019.
Application No. 16/399,988 is a continuation-in-part of application No. 16/454,865, filed on Jun. 27, 2019.
(60) Provisional application No. 62/718,449, filed on Aug. 14, 2018.
(60) Provisional application No. 62/735,365, filed on Sep. 24, 2018.
(60) Provisional application No. 62/781,112, filed on Dec. 18, 2018.
(60) Provisional application No. 62/779,119, filed on Dec. 13, 2018.
(60) Provisional application No. 62/841,213, filed on Apr. 30, 2019.

(51) Int. Cl. C12N 15/70; C12M 3/00; C12M 1/42; C12M 1/12; C12N 15/90

(52) U.S. Cl. CPC C12N 15/70; C12M 23/44; C12M 35/02; C12N 2800/80; C12N 15/90; C12N 2310/20; C12M 37/02

(57) ABSTRACT

The present disclosure provides methods, automated modules, and instruments for enrichment of live cells that have been edited by nucleic acid-guided nuclease genome editing. The disclosure provides improved methods and modules—including high throughput methods and modules—for enriching for cells that have been subjected to editing.

### RELATED APPLICATIONS

This application claims priority to US Provisional Application Nos: 62/718,449, filed 14 Aug. 2018; 62/735,365, filed 24 Sep. 2018; 62/781,112, filed 18 Dec. 2018; 62/779,119, filed 13 Dec. 2018; 62/841,213, filed 30 Apr. 2019. This application is Continuation In Part of U.S. Utility application Ser. No. 16/399,988, filed 30 Apr. 2019; and Ser. No. 16/454,865, filed 27 Jun. 2019.

### FIELD OF THE INVENTION

This invention relates to automated modules and systems for culturing and editing live cells via bulk cell culture.

### BACKGROUND OF THE INVENTION

In the following discussion certain articles and methods will be described for background and introductory purposes. Nothing contained herein is to be construed as an “admission” of prior art. Applicant expressly reserves the right to demonstrate, where appropriate, that the methods referenced herein do not constitute prior art under the applicable statutory provisions.

The ability to make precise, targeted changes to the genome of living cells has been a long-standing goal in biomedical research and development. Recently, various nucleases have been identified that allow manipulation of gene sequence, and hence gene function. The nucleases include nucleic acid-guided nucleases, which enable researchers to generate permanent edits in live cells. Editing efficiencies in cell populations can be high; however, in pooled or multiplex formats there tends to be selective enrichment of cells that have not been edited due to the lack of the double-strand. DNA breaks that occur during the editing process. Double-strand DNA breaks dramatically negatively impact cell viability thereby leading to enhanced survival of unedited cells and making it difficult to identify edited cells in the background of unedited cells. In addition, cells with edits that confer growth advantages or disadvantages can lead to skewed representations for different edits in the population.

There is thus a need in the art of nucleic acid-guided nuclease gene editing for improved methods for enriching for cells that have been edited. The present invention satisfies this need.

### SUMMARY OF THE INVENTION

This Summary is provided to introduce a selection of concepts in a simplified form that are further described below in the Detailed Description. This Summary is not intended to identify key or essential features of the claimed subject matter, nor is it intended to be used to limit the scope of the claimed subject matter. Other features, details, utilities, and advantages of the claimed subject matter will be apparent from the following written Detailed Description including those aspects illustrated in the accompanying drawings and defined in the appended claims.

The present disclosure provides methods, modules, instruments and systems for automated high-throughput and extremely sensitive enriching for cells edited by a nucleic acid-guided nuclease. The methods take advantage of isolation or singulation, where the term in this context refers to the process of separating cells and growing them into clonal colonies. Isolation (or singulation) overcomes growth bias from unedited cells, growth effects from differential editing rates, and growth bias resulting from fitness effects of different edits. Indeed, it has been determined that removing growth rate bias via isolation or substantial isolation and growing colonies from the isolated cells to saturation or terminal colony size (e.g., normalization of the colonies) improves the observed editing efficiency by up to 4× or more over conventional methods. One particularly facile method for isolation utilizes a bulk cell culture format, which is described in detail herein.

Thus in one embodiment there is provided a method for performing enrichment of cells edited by a nucleic acid-guided nuclease, comprising: providing transformed cells at a dilution resulting in substantially cells in an appropriate liquid growth medium comprising 0.25%-6% alginate, wherein the cells comprise nucleic acid-guided nuclease editing components where the gRNA optionally is under the control of an inducible promoter; solidifying the alginate-containing medium with a divalent cation; allowing the isolated cells to grow for 2 to 50 doublings to establish cell colonies; optionally inducing transcription of the gRNA; allowing the cell colonies to grow to become normalized; and liquefying the alginate-containing medium with a divalent cation chelating agent. In some aspects, the nucleic acid-guided nuclease editing components are provided to the cells on two separate vectors and in some aspects, the nucleic acid-guided nuclease editing components are provided to the cells on a single vector, and in some aspects, the cells are bacterial cells, yeast cells, or mammalian cells.

In some aspects of this method embodiment, the percentage of alginate in the growth medium is 1%-4%, and in some aspects, the percentage of alginate in the growth medium is 2%-3%.

In some aspects, the inducible promoter driving the gRNA is a promoter that is activated upon an increase in temperature, and in some aspects, the inducible promoter is a pL promoter, the cells are transformed with a coding sequence for the CI857 repressor, and transcription of the one or more nucleic acid-guided nuclease editing components is induced by raising temperature of the cells to 42° C.

In some aspects, solidifying the alginate-containing medium is performed with divalent cations except Mg⁺², and in some embodiments, the divalent cation is Ca⁺². In some aspects, the divalent cation chelating agent (e.g., liquefying agent) is citrate, ethylenediaminetetraacetic acid (EDTA), or hexametaphosphate.

Also provided is a module for performing automated enrichment of cells edited by a nucleic acid-guided nuclease editing comprising: means for providing cells transformed with one or more vectors comprising a coding sequence for a nuclease, a guide nucleic acid and a DNA donor sequence; means for diluting the transformed cells in a medium comprising 0.25% to 6% (w/v) alginate to a cell density appropriate to isolate the transformed cells in a vessel; means for solidifying the alginate-containing medium; means for providing a temperature to grow the cells; and means for re-liquefying the solidified alginate-containing medium with a chelating agent for a divalent cation.

In some aspects, there is provided a multi-module cell editing instrument comprising the module for performing automated enrichment of cells edited by a nucleic acid-guided nuclease, where the multi-module cell editing instrument further comprises a liquid handling system for providing cells, diluting cells, dispensing a solidifying agent, and dispensing a liquefying agent. Also in some aspects of a multi-module cell editing instrument comprising the module for performing automated enrichment of cells edited by a nucleic acid-guided nuclease, there is further provided a transformation module, and/or a growth module, and/or a nucleic acid assembly module, and/or a reagent cartridge. In some aspects, when present the reagent cartridge comprises CaCl₂ and Na₃C₆H₅O₇. In some specific embodiments of the multi-module cell editing instrument comprising the module for performing automated enrichment of cells, a means to control temperature of the rotating growth vial and its contents, where the transformation module comprises a flow-through electroporation device.

In other aspects there is provided a method for performing enrichment of cells edited by a nucleic acid-guided nuclease comprising providing transformed cells at a dilution in a vessel resulting in isolated cells in an appropriate liquid growth medium comprising a hydrogel, wherein the cells comprise a gRNA under the control of an inducible promoter; solidifying the hydrogel-containing medium with a solution of a divalent cation; allowing the isolated cells to grow for 2 and 50 doublings to establish cell colonies; inducing transcription of the gRNA; growing the cells for a period of time sufficient to allow the cell colonies to become normalized; and liquefying the hydrogel-containing medium with a chelating agent for the divalent cation. There is also provided a module to perform this method, and a multi-module cell editing instrument comprising the module. In some aspects, the hydrogel-containing medium may be liquefied by agitation of the gel by, e.g., beads.

In aspects of the embodiment, the inducible promoter is a temperature inducible promoter, and the means for providing a temperature to induce transcription of the nuclease or guide nucleic acid is a Peltier device. In some aspects of this embodiment, solidifying the alginate-containing medium is performed with divalent cations except Mg⁺², and in some embodiments, the divalent cation is Ca⁺². In some aspects, the divalent cation chelating agent (e.g., liquefying agent) is citrate, ethylenediaminetetraacetic acid (EDTA), or hexametaphosphate.

These aspects and other features and advantages of the invention are described below in more detail.

### BRIEF DESCRIPTION OF THE FIGURES

FIG. 1A is a simplified flow chart of two exemplary methods that may be performed by an automated bulk cell culture module, either as a stand-alone instrument or as part of an automated multi-module cell processing instrument. FIG. 1B is a plot of optical density vs. time showing the growth curves for edited cells (dotted line) and unedited cells (solid line).

FIG. 2A depicts a simplified graphic for a workflow for isolating, editing and normalizing cells after nucleic acid-guided nuclease genome editing in bulk cell culture, where reversible solidification of the bulk culture is utilized. FIG. 2B depicts a simplified graphic for a workflow for isolating, editing and normalizing cells after nucleic acid-guided nuclease genome editing in bulk cell culture, where reversible solidification of the bulk culture is utilized, and editing is induced by inducing transcription of gRNA. FIG. 2C is a photograph of E. coli cells expressing green fluorescent protein in 2.0% alginate and medium that has been solidified showing isolated colonies (left) and a photograph of E. coli cells expressing green fluorescent protein in 2.0% alginate and medium after the medium has been re-liquified.

FIG. 3A depicts an automated multi-module cell processing instrument. FIGS. 3B-3D depict a reagent cartridge and a flow-through electroporation device that is configured to reside in the reagent cartridge. FIGS. 3E-3L depict various components of one embodiment of a tangential flow filtration device which serves as a cell concentration and buffer exchange module in the automated multi-module cell processing instrument shown in FIG. 3A.

FIG. 4A depicts one embodiment of a rotating growth vial for use with a cell growth module described herein. FIG. 4B illustrates a perspective view of one embodiment of a cell growth module comprising a rotating growth vial housing. FIG. 4C depicts a cut-away view of the cell growth module from FIG. 4B. FIG. 4D illustrates the cell growth module of FIG. 4B coupled to LED, detector, and temperature regulating components.

FIG. 5A is a simplified block diagram of an embodiment of an exemplary automated multi-module cell processing instrument comprising a bulk gel isolation/growth/editing/normalization module. FIG. 5B is a simplified block diagram of an alternative embodiment of an exemplary automated multi-module cell processing instrument comprising a bulk gel isolation/growth/editing/normalization module.

FIGS. 6A-6C depict a process for determining whether normalization takes place when cells are cultured in bulk gel.

FIG. 7 is a photograph of a bulk gel cell culture workflow for automation in a bulk gel isolation/growth/editing/normalization module utilizing a rotating growth vial, such as that depicted in FIG. 4A.

FIGS. 8A, 8B, and 8C depict a graph, table, and two graphs, respectively, of the results obtained from editing experiments performed with liquid cell culture employing no isolation or normalization, but employing inducible editing; bulk cell gel culture, employing isolation, inducible editing, and normalization; solid agar plating (SPP) employing isolation, inducible editing, and normalization; solid agar plating (SPP-Cherry) employing isolation, inducible editing, and cherry picking; and solid agar plating (SPP) employing isolation, inducible editing, and normalization but without cherry picking and simply scraping the colonies from the plate and re-plating.

FIG. 9 depicts a recursive workflow using bulk gel cell culture with curing.

### DETAILED DESCRIPTION

All of the functionalities described in connection with one embodiment are intended to be applicable to the additional embodiments described herein except where expressly stated or where the feature or function is incompatible with the additional embodiments. For example, where a given feature or function is expressly described in connection with one embodiment but not expressly mentioned in connection with an alternative embodiment, it should be understood that the feature or function may be deployed, utilized, or implemented in connection with the alternative embodiment unless the feature or function is incompatible with the alternative embodiment.

The practice of the techniques described herein may employ, unless otherwise indicated, conventional techniques and descriptions of organic chemistry, polymer technology, molecular biology (including recombinant techniques), cell biology, biochemistry, and sequencing technology, which are within the skill of those who practice in the art. Such conventional techniques include polymer array synthesis, hybridization and ligation of polynucleotides, and detection of hybridization using a label. Specific illustrations of suitable techniques can be had by reference to the examples herein. However, other equivalent conventional procedures can, of course, also be used. Such conventional techniques and descriptions can be found in standard laboratory manuals such as Green, et al., eds., Genome Analysis: A Laboratory Manual Series (Vols. I-IV) (1999); Weiner, Gabriel, Stephens, eds., Genetic Variation: A Laboratory Manual (2007); Dieffenbach, Dveksler, eds., PCR Primer: A Laboratory Manual (2003); Bowtell and Sambrook, DNA Microarrays: A Molecular Cloning Manual (2003); Mount, Bioinformatics: Sequence and Genome Analysis (2004); Sambrook and Russell, Condensed Protocols from Molecular Cloning: A Laboratory Manual (2006); Stryer, Biochemistry (4th Ed.) W.H. Freeman, New York N.Y. (1995); Gait, “Oligonucleotide Synthesis: A Practical Approach” (1984), IRL Press, London; Nelson and Cox, Lehninger, Principles of Biochemistry 3^{rd} Ed., W. H. Freeman Pub., New York, N.Y. (2000); Berg et al., Biochemistry, 5^{th} Ed., W.H. Freeman Pub., New York, N.Y. (2002); Doyle & Griffiths, eds., Cell and Tissue Culture: Laboratory Procedures in Biotechnology, Doyle & Griffiths, eds., John Wiley & Sons (1998); G. Hadlaczky, ed. Mammalian Chromosome Engineering—Methods and Protocols, Humana Press (2011); and Lanza and Klimanskaya, eds., Essential Stem Cell Methods, Academic Press (2011), all of which are herein incorporated in their entirety by reference for all purposes. CRISPR-specific techniques can be found in, e.g., Appasani and Church, Genome Editing and Engineering From TALENs and CRISPRs to Molecular Surgery (2018); and Lindgren and Charpentier, CRISPR: Methods and Protocols (2015); both of which are herein incorporated in their entirety by reference for all purposes.

Note that as used herein and in the appended claims, the singular forms “a,” “an,” and “the” include plural referents unless the context clearly dictates otherwise. Thus, for example, reference to “a cell” refers to one or more cells, and reference to “the system” includes reference to equivalent steps, methods and devices known to those skilled in the art, and so forth. Additionally, it is to be understood that terms such as “left,” “right,” “top,” “bottom,” “front,” “rear,” “side,” “height,” “length,” “width,” “upper,” “lower,” “interior,” “exterior,” “inner,” and/or “outer” that may be used herein merely describe points of reference and do not necessarily limit embodiments of the present disclosure to any particular orientation or configuration. Furthermore, terms such as “first,” “second,” “third,” etc., merely identify one of a number of portions, components, steps, operations, functions, and/or points of reference as disclosed herein, and likewise do not necessarily limit embodiments of the present disclosure to any particular configuration or orientation.

Additionally, the terms “approximately,” “proximate,” “minor,” and similar terms generally refer to ranges that include the identified value within a margin of 20%, 10% or preferably 5% in certain embodiments, and any values therebetween.

Unless defined otherwise, all technical and scientific terms used herein have the same meaning as commonly understood by one of ordinary skill in the art to which this invention belongs. All publications mentioned herein are incorporated by reference for the purpose of describing and disclosing devices, methods and cell populations that may be used in connection with the presently described invention.

Where a range of values is provided, it is understood that each intervening value, between the upper and lower limit of that range and any other stated or intervening value in that stated range is encompassed within the invention. The upper and lower limits of these smaller ranges may independently be included in the smaller ranges, and are also encompassed within the invention, subject to any specifically excluded limit in the stated range. Where the stated range includes one or both of the limits, ranges excluding either both of those included limits are also included in the invention.

In the following description, numerous specific details are set forth to provide a more thorough understanding of the present invention. However, it will be apparent to one of ordinary skill in the art that the present invention may be practiced without one or more of these specific details. In other instances, features and procedures well known to those skilled in the art have not been described in order to avoid obscuring the invention.

The term “complementary” as used herein refers to Watson-Crick base pairing between nucleotides and specifically refers to nucleotides that are hydrogen bonded to one another with thymine or uracil residues linked to adenine residues by two hydrogen bonds and cytosine and guanine residues linked by three hydrogen bonds. In general, a nucleic acid includes a nucleotide sequence described as having a “percent complementarity” or “percent homology” to a specified second nucleotide sequence. For example, a nucleotide sequence may have 80%, 90%, or 100% complementarity to a specified second nucleotide sequence, indicating that 8 of 10, 9 of 10 or 10 of 10 nucleotides of a sequence are complementary to the specified second nucleotide sequence. For instance, the nucleotide sequence 3′-TCGA-5′ is 100% complementary to the nucleotide sequence 5′-AGCT-3′; and the nucleotide sequence 3′-TCGA-5′ is 100% complementary to a region of the nucleotide sequence 5′-TTAGCTGG-3′.

The term DNA “control sequences” refers collectively to promoter sequences, polyadenylation signals, transcription termination sequences, upstream regulatory domains, origins of replication, internal ribosome entry sites, nuclear localization sequences, enhancers, and the like, which collectively provide for the replication, transcription and translation of a coding sequence in a recipient cell. Not all of these types of control sequences need to be present so long as a selected coding sequence is capable of being replicated, transcribed and—for some components—translated in an appropriate host cell.

As used herein the term “donor DNA” or “donor nucleic acid” refers to nucleic acid that is designed to introduce a DNA sequence modification (insertion, deletion, substitution) into a locus by homologous recombination using nucleic acid-guided nucleases. For homology-directed repair, the donor DNA must have sufficient homology to the regions flanking the “cut site” or site to be edited in the genomic target sequence. The length of the homology arm(s) will depend on, e.g., the type and size of the modification being made. For example, the donor DNA will have at least one region of sequence homology (e.g., one homology arm) to the genomic target locus. In many instances and preferably, the donor DNA will have two regions of sequence homology (e.g., two homology arms) to the genomic target locus. Preferably, an “insert” region or “DNA sequence modification” region—the nucleic acid modification that one desires to be introduced into a genome target locus in a cell—will be located between two regions of homology. The DNA sequence modification may change one or more bases of the target genomic DNA sequence at one specific site or multiple specific sites. A change may include changing 1, 2, 3, 4, 5, 10, 15, 20, 25, 30, 35, 40, 50, 75, 100, 150, 200, 300, 400, or 500 or more base pairs of the target sequence. A deletion or insertion may be a deletion or insertion of 1, 2, 3, 4, 5, 10, 15, 20, 25, 30, 35, 40, 50, 75, 100, 150, 200, 300, 400, or 500 or more base pairs of the target sequence. The donor DNA optionally further includes an alteration to the target sequence, e.g., a PAM mutation that prevents binding of the nuclease at the PAM or spacer in the target sequence after editing has taken place.

As used herein, “enrichment” refers to enriching for edited cells by isolation or substantial isolation of cells, initial growth of cells into cell colonies (e.g., incubation), editing (optionally induced, particularly in bacterial systems), and growing the cell colonies into terminal-sized colonies (e.g., saturation or normalization of colony growth).

The terms “guide nucleic acid” or “guide RNA” or “gRNA” refer to a polynucleotide comprising 1) a guide sequence capable of hybridizing to a genomic target locus, and 2) a scaffold sequence capable of interacting or complexing with a nucleic acid-guided nuclease.

“Homology” or “identity” or “similarity” refers to sequence similarity between two peptides or, more often in the context of the present disclosure, between two nucleic acid molecules. The term “homologous region” or “homology arm” refers to a region on the donor DNA with a certain degree of homology with the target genomic DNA sequence. Homology can be determined by comparing a position in each sequence which may be aligned for purposes of comparison. When a position in the compared sequence is occupied by the same base or amino acid, then the molecules are homologous at that position. A degree of homology between sequences is a function of the number of matching or homologous positions shared by the sequences.

As used herein, the terms “isolation” or “isolate” mean to separate individual cells so that each cell (and the colonies formed from each cell) will be separate from other cells; for example, a single cell in a single microwell, or 100 single cells each in its own microwell. “Isolation” or “isolated cells” result in one embodiment, from a Poisson distribution in arraying cells. The terms “substantially isolated”, “largely isolated”, and “substantial isolation” mean cells are largely separated from one another, in small groups or batches. That is, when 2, 3, 4, 5, 6, 7, 8, 9, 10, 11, 12, 13, 14, 15, 16, 17, 18, 19, 20, 21, 22, 23, 24, 25, 26, 27, 28, 29, 30 or up to 50—but preferably 10 or less cells—are delivered to a microwell. “Substantially isolated” or “largely isolated” result, in one embodiment, from a “substantial Poisson distribution” in arraying cells. With more complex libraries of edits—or with libraries that may comprise lethal edits or edits with greatly-varying fitness effects—it is preferred that cells be isolated via a Poisson distribution.

“Operably linked” refers to an arrangement of elements where the components so described are configured so as to perform their usual function. Thus, control sequences operably linked to a coding sequence are capable of effecting the transcription, and in some cases, the translation, of a coding sequence. The control sequences need not be contiguous with the coding sequence so long as they function to direct the expression of the coding sequence. Thus, for example, intervening untranslated yet transcribed sequences can be present between a promoter sequence and the coding sequence and the promoter sequence can still be considered “operably linked” to the coding sequence. In fact, such sequences need not reside on the same contiguous DNA molecule (i.e. chromosome) and may still have interactions resulting in altered regulation.

A “promoter” or “promoter sequence” is a DNA regulatory region capable of binding RNA polymerase and initiating transcription of a polynucleotide or polypeptide coding sequence such as messenger RNA, ribosomal RNA, small nuclear or nucleolar RNA, guide RNA, or any kind of RNA transcribed by any class of any RNA polymerase I, II or III. Promoters may be constitutive or inducible. In the methods described herein optionally the promoters driving transcription of the gRNAs is inducible.

As used herein the term “selectable marker” refers to a gene introduced into a cell, which confers a trait suitable for artificial selection. General use selectable markers are well-known to those of ordinary skill in the art. Drug selectable markers such as ampilcillin/carbenicillin, kanamycin, chloramphenicol, erythromycin, tetracycline, gentamicin, bleomycin, streptomycin, puromycin, hygromycin, blasticidin, and G418 may be employed. In other embodiments, selectable markers include, but are not limited to human nerve growth factor receptor (detected with a MAb, such as described in U.S. Pat. No. 6,365,373); truncated human growth factor receptor (detected with MAb); mutant human dihydrofolate reductase (DHFR; fluorescent MTX substrate available); secreted alkaline phosphatase (SEAP; fluorescent substrate available); human thymidylate synthase (TS; confers resistance to anti-cancer agent fluorodeoxyuridine); human glutathione S-transferase alpha (GSTA1; conjugates glutathione to the stem cell selective alkylator busulfan; chemoprotective selectable marker in CD34+cells); CD24 cell surface antigen in hematopoietic stem cells; rhamnose; human CAD gene to confer resistance to N-phosphonacetyl-L-aspartate (PALA); human multi-drug resistance-1 (MDR-1; P-glycoprotein surface protein selectable by increased drug resistance or enriched by FACS); human CD25 (IL-2a; detectable by MAb-FITC); Methylguanine-DNA methyltransferase (MGMT; selectable by carmustine); and Cytidine deaminase (CD; selectable by Ara-C). “Selective medium” as used herein refers to cell growth medium to which has been added a chemical compound or biological moiety that selects for or against selectable markers.

The terms “target genomic DNA sequence”, “target sequence”, or “genomic target locus” refer to any locus in vitro or in vivo, or in a nucleic acid (e.g., genome) of a cell or population of cells, in which a change of at least one nucleotide is desired using a nucleic acid-guided nuclease editing system. The target sequence can be a genomic locus or extrachromosomal locus.

A “vector” is any of a variety of nucleic acids that comprise a desired sequence or sequences to be delivered to and/or expressed in a cell. Vectors are typically composed of DNA, although RNA vectors are also available. Vectors include, but are not limited to, plasmids, fosmids, phagemids, virus genomes, YACs, BACs, mammalian synthetic chromosomes, and the like. As used herein, the phrase “engine vector” comprises a coding sequence for a nuclease—optionally under the control of an inducible promoter—to be used in the nucleic acid-guided nuclease systems and methods of the present disclosure. The engine vector may also comprise, in a bacterial system, the λ Red recombineering system or an equivalent thereto, as well as a selectable marker. As used herein the phrase “editing vector” comprises a donor nucleic acid, including an alteration to the target sequence which prevents nuclease binding at a PAM or spacer in the target sequence after editing has taken place, and a coding sequence for a gRNA optionally under the control of an inducible promoter (and preferably under the control of an inducible promoter in bacterial systems). The editing vector may also comprise a selectable marker and/or a barcode. In some embodiments, the engine vector and editing vector may be combined; that is, the contents of the engine vector may be found on the editing vector.

### Editing in Nucleic Acid-Guided Nuclease Genome Systems Generally

The present disclosure provides instruments, modules and methods for nucleic acid-guided nuclease genome editing that provide 1) enhanced observed editing efficiency of nucleic acid-guided nuclease editing methods, and 2) improvement in enriching for cells whose genomes have been properly edited, including high-throughput screening techniques. Presented herein are methods that take advantage of isolation (separating cells and growing them into clonal colonies) and normalization. Isolation or substantial isolation, incubation, followed by editing (optionally with a gRNA under the control of an inducible promoter) and normalization overcomes growth bias from unedited cells. The instruments, modules, and methods may be applied to all cell types including, archaeal, prokaryotic, and eukaryotic (e.g., yeast, fungal, plant and animal) cells. Most normal mammalian tissue-derived cells—except those derived from the hematopoietic system—are anchorage dependent and need a surface or cell culture support for normal proliferation. Adherent cells may be grown on beads that are isolated in the bulk gel. Cell culture beads or scaffolds appropriate for this purpose typically have a diameter of 100-300 μm and have a density slightly greater than that of the culture medium (here, the liquefied culture medium thus facilitating an easy separation of cells and medium for, e.g., medium exchange) yet the density must also be sufficiently low to allow complete suspension of the carriers at a minimum stirring rate in order to avoid hydrodynamic damage to the cells. Many different types of microcarriers are available, and different microcarriers are optimized for different types of cells. There are positively charged carriers, such as Cytodex 1 (dextran-based, GE Healthcare), DE-52 (cellulose-based, Sigma-Aldrich Labware), DE-53 (cellulose-based, Sigma-Aldrich Labware), HLX 11-170 (polystyrene-based); collagen or ECM (extracellular matrix)-coated carriers, such as Cytodex 3 (dextran-based, GE Healthcare) or HyQ-sphere Pro-F 102-4 (polystyrene-based, Thermo Scientific); non-charged carriers, like HyQspheres P 102-4 (Thermo Scientific); or macroporous carriers based on gelatin (Cultisphere, Percell Biolytica) or cellulose (Cytopore, GE Healthcare).

The instruments, modules, and methods described herein employ editing cassettes comprising a guide RNA (gRNA) sequence covalently linked to a donor DNA sequence where, particularly in bacterial systems, the gRNA optionally is under the control of an inducible promoter (e.g., the editing cassettes are CREATE cassettes; see U.S. patent Ser. No. 9/982,278, issued 29 May 2019 and Ser. No. 10/240,167, issued 26 Mar. 2019; Ser. No. 10/266,849, issued 23 Apr. 2019; and U.S. Ser. No. 15/948,785, filed 9 Apr. 2018; Ser. No. 16/275,439, filed 14 Feb. 2019; and Ser. No. 16/275,465, filed 14 Feb. 2019, all of which are incorporated by reference in their entirety). The disclosed methods allow for cells to be transformed, substantially isolated, grown for several doublings (e.g., incubation), after which editing is allowed. The isolation process effectively negates the effect of unedited cells taking over the cell population. The combination of substantially isolating cells, then allowing for initial growth followed by optionally inducing transcription of the gRNA (and optionally the nuclease) and normalization of cell colonies leads to 2-250×, 10-225×, 25-200×, 40-175×, 50-150×, 60-100×, or 50-100× gains in identifying edited cells over prior art methods and allows for generation of arrayed or pooled edited cells comprising cell libraries with edited genomes. Additionally, the methods may be leveraged to create iterative editing systems to generate combinatorial libraries of cells with two to many edits in each cellular genome.

The instruments, compositions and methods described herein improve editing systems in which nucleic acid-guided nucleases (e.g., RNA-guided nucleases) are used to edit specific target regions in an organism's genome. A nucleic acid-guided nuclease complexed with an appropriate synthetic guide nucleic acid in a cell can cut the genome of the cell at a desired location. The guide nucleic acid helps the nucleic acid-guided nuclease recognize and cut the DNA at a specific target sequence. By manipulating the nucleotide sequence of the guide nucleic acid, the nucleic acid-guided nuclease may be programmed to target any DNA sequence for cleavage as long as an appropriate protospacer adjacent motif (PAM) is nearby. In certain aspects, the nucleic acid-guided nuclease editing system may use two separate guide nucleic acid molecules that combine to function as a guide nucleic acid, e.g., a CRISPR RNA (crRNA) and trans-activating CRISPR RNA (tracrRNA). In other aspects, the guide nucleic acid may be a single guide nucleic acid that includes both the crRNA and tracrRNA sequences or a single guide nucleic acid that does not require a tracrRNA.

In general, a guide nucleic acid (e.g., gRNA) complexes with a compatible nucleic acid-guided nuclease and can then hybridize with a target sequence, thereby directing the nuclease to the target sequence. A guide nucleic acid can be DNA or RNA; alternatively, a guide nucleic acid may comprise both DNA and RNA. In some embodiments, a guide nucleic acid may comprise modified or non-naturally occurring nucleotides. In cases where the guide nucleic acid comprises RNA, the gRNA is encoded by a DNA sequence on a polynucleotide molecule such as a plasmid, linear construct, or resides within an editing cassette and is optionally-particularly in bacterial systems-under the control of an inducible promoter.

A guide nucleic acid comprises a guide sequence, where the guide sequence is a polynucleotide sequence having sufficient complementarity with a target sequence to hybridize with the target sequence and direct sequence-specific binding of a complexed nucleic acid-guided nuclease to the target sequence. The degree of complementarity between a guide sequence and the corresponding target sequence, when optimally aligned using a suitable alignment algorithm, is about or more than about 50%, 60%, 75%, 80%, 85%, 90%, 95%, 97.5%, 99%, or more. Optimal alignment may be determined with the use of any suitable algorithm for aligning sequences. In some embodiments, a guide sequence (the portion of the guide nucleic acid that hybridizes with the target sequence) is about or more than about 10, 11, 12, 13, 14, 15, 16, 17, 18, 19, 20, 21, 22, 23, 24, 25, 26, 27, 28, 29, 30, 35, 40, 45, 50, 75, or more nucleotides in length. In some embodiments, a guide sequence is less than about 75, 50, 45, 40, 35, 30, 25, 20 nucleotides in length. Preferably the guide sequence is 10-30 or 15-20 nucleotides long, or 15, 16, 17, 18, 19, or 20 nucleotides in length.

In the present methods and compositions, the guide nucleic acid is provided as a sequence to be expressed from a plasmid or vector and comprises both the guide sequence and the scaffold sequence as a single transcript. Alternatively, the guide nucleic acids may be transcribed from two separate sequences. The guide nucleic acid can be engineered to target a desired target DNA sequence by altering the guide sequence so that the guide sequence is complementary to the target DNA sequence, thereby allowing hybridization between the guide sequence and the target DNA sequence. In general, to generate an edit in the target DNA sequence, the gRNA/nuclease complex binds to a target sequence as determined by the guide RNA, and the nuclease recognizes a protospaeer adjacent motif (PAM) sequence adjacent to the target sequence. The target sequence can be any polynucleotide (either DNA or RNA) endogenous or exogenous to a prokaryotic or eukaryotic cell, or in vitro. For example, the target sequence can be a polynucleotide residing in the nucleus of a eukaryotic cell. A target sequence can be a sequence encoding a gene product (e.g., a protein) and/or a non-coding sequence (e.g., a regulatory polynucleotide, an intron, a PAM, or “junk” DNA).

The guide nucleic acid may be part of an editing cassette that encodes the donor nucleic acid; that is, the editing cassette may be a CREATE cassette (see, e.g U.S. patent Ser. No. 9/982,278, issued 29 May 2019 and Ser. No. 10/240,167, issued 26 Mar. 2019 Ser. No. 10/266,849, issued 23 Apr. 2019; and U.S. Ser. No. 15/948,785, filed 9 Apr. 2018; Ser. No. 16/275,439, filed 14 Feb. 2019; and Ser. No. 16/275,465, filed 14 Feb. 2019, all of which are incorporated by reference in their entirety). The guide nucleic acid and the donor nucleic acid may be and typically are under the control of a single (optionally inducible) promoter. Alternatively, the guide nucleic acid may not be part of the editing cassette and instead may be encoded on the engine or editing vector backbone. For example, a sequence coding for a guide nucleic acid can be assembled or inserted into a vector backbone first, followed by insertion of the donor nucleic acid. In other cases, the donor nucleic acid can be inserted or assembled into a vector backbone first, followed by insertion of the sequence coding for the guide nucleic acid. In yet other cases, the sequence encoding the guide nucleic acid and the donor nucleic acid (inserted, for example, in an editing cassette) are simultaneously but separately inserted or assembled into a vector. In yet other embodiments and preferably, the sequence encoding the guide nucleic acid and the sequence encoding the donor nucleic acid are both included in the editing cassette.

The target sequence is associated with a PAM, which is a short nucleotide sequence recognized by the gRNA/nuclease complex. The precise PAM sequence and length requirements for different nucleic acid-guided nucleases vary; however, PAMs typically are 2-7 base-pair sequences adjacent or in proximity to the target sequence and, depending on the nuclease, can be 5′ or 3′ to the target sequence. Engineering of the PAM-interacting domain of a nucleic acid-guided nuclease may allow for alteration of PAM specificity, improve target site recognition fidelity, decrease target site recognition fidelity, and increase the versatility of a nucleic acid-guided nuclease. In certain embodiments, the genome editing of a target sequence both introduces a desired DNA change to a target sequence, e.g., the genomic DNA of a cell, and removes, mutates, or renders inactive a proto-spacer (PAM) region in the target sequence; that is, the donor DNA often includes an alteration to the target sequence that prevents binding of the nuclease at the PAM in the target sequence after editing has taken place. Rendering the PAM at the target sequence inactive precludes additional editing of the cell genome at that target sequence, e.g., upon subsequent exposure to a nucleic acid-guided nuclease complexed with a synthetic guide nucleic acid in later rounds of editing. Thus, cells having the desired target sequence edit and an altered PAM can be selected using a nucleic acid-guided nuclease complexed with a synthetic guide nucleic acid complementary to the target sequence. Cells that did not undergo the first editing event will be cut rendering a double-stranded DNA break, and thus will not continue to be viable. The cells containing the desired target sequence edit and PAM alteration will not be cut, as these edited cells no longer contain the necessary PAM site and will continue to grow and propagate.

The range of target sequences that nucleic acid-guided nucleases can recognize is constrained by the need for a specific PAM to be located near the desired target sequence. As a result, it often can be difficult to target edits with the precision that is necessary for genome editing. It has been found that nucleases can recognize some PAMs very well (e.g., canonical PAMs), and other PAMs less well or poorly (e.g., non-canonical PAMs). Because the methods disclosed herein allow for identification of edited cells in a large background of unedited cells, the methods allow for identification of edited cells where the PAM is less than optimal; that is, the methods for identifying edited cells herein allow for identification of edited cells even if editing efficiency is very low. Additionally, the present methods expand the scope of target sequences that may be edited since edits are more readily identified, including cells where the genome edits are associated with less functional PAMs.

As for the nuclease component of the nucleic acid-guided nuclease editing system, the polynucleotide sequence encoding the nucleic acid-guided nuclease can be codon optimized for expression in particular cells, such as archaeal, prokaryotic or eukaryotic cells. Eukaryotic cells can be yeast, fungi, algae, plant, animal, or human cells. Eukaryotic cells may be those of or derived from a particular organism, such as a mammal, including but not limited to human, mouse, rat, rabbit, dog, or non-human mammal including non-human primate. The choice of nucleic acid-guided nuclease to be employed depends on many factors, such as what type of edit is to be made in the target sequence and whether an appropriate PAM is located close to the desired target sequence. Nucleases of use in the methods described herein include but are not limited to Cas 9, Cas 12/CpfI, MAD2, or MAD7 or other MADzymes. As with the guide nucleic acid, the nuclease may be encoded by a DNA sequence on a vector (e.g., the engine vector) and be under the control of a constitutive or an inducible promoter. Again, at least one of and preferably both of the nuclease and guide nucleic acid are under the control of an inducible promoter.

Another component of the nucleic acid-guided nuclease system is the donor nucleic acid. In some embodiments, the donor nucleic acid is on the same polynucleotide (e.g., vector or editing (CREATE) cassette) as the guide nucleic acid. The donor nucleic acid is designed to serve as a template for homologous recombination with a target sequence nicked or cleaved by the nucleic acid-guided nuclease as a part of the gRNA/nuclease complex. A donor nucleic acid polynucleotide may be of any suitable length, such as about or more than about 30, 35, 40, 45, 50, 75, 100, 150, 200, 500, 1000, 2500, 5000 nucleotides or more in length. In certain preferred aspects, the donor nucleic acid can be provided as an oligonucleotide of between 40-300 nucleotides, more preferably between 50-250 nucleotides. The donor nucleic acid comprises a region that is complementary to a portion of the target sequence (e.g., a homology arm). When optimally aligned, the donor nucleic acid overlaps with (is complementary to) the target sequence by, e.g., about 10, 15, 20, 25, 30, 35, 40, 50, 60, 70, 80, 90 or more nucleotides. In many embodiments, the donor nucleic acid comprises two homology arms (regions complementary to the target sequence) flanking the mutation or difference between the donor nucleic acid and the target template. The donor nucleic acid comprises at least one mutation or alteration compared to the target sequence, such as an insertion, deletion, modification, or any combination thereof compared to the target sequence.

Often the donor nucleic acid is provided as an editing cassette, which is inserted into a vector backbone where the vector backbone may comprise a promoter driving transcription of the gRNA and the donor nucleic acid. Moreover, there may be more than one, e.g., two, three, four, or more guide nucleic acid/donor nucleic acid cassettes inserted into an engine vector, where the guide nucleic acids are under the control of separate, different promoters, separate, like promoters, or where all guide nucleic acid/donor nucleic acid pairs are under the control of a single promoter. (See, e.g., U.S. Ser. No. 16/275,465, filed 14 Feb. 2019, drawn to multiple CREATE cassettes.) The promoter driving transcription of the gRNA and the donor nucleic acid (or driving more than one gRNA/donor nucleic acid pair) is optionally an inducible promoter (and in bacterial systems is preferably an inducible promoter) and the promoter driving transcription of the nuclease is optionally an inducible promoter as well.

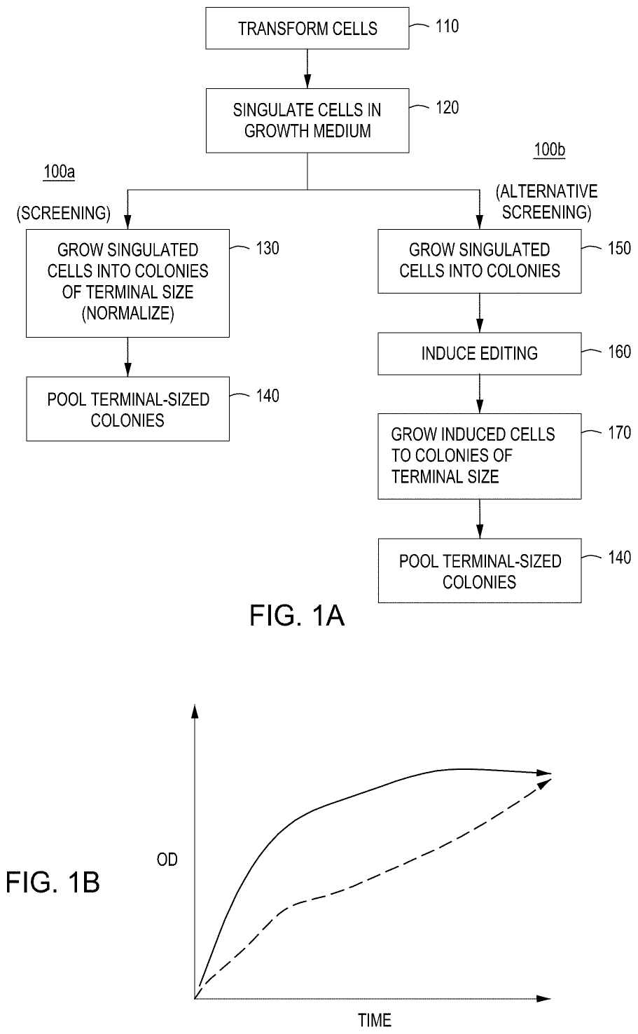

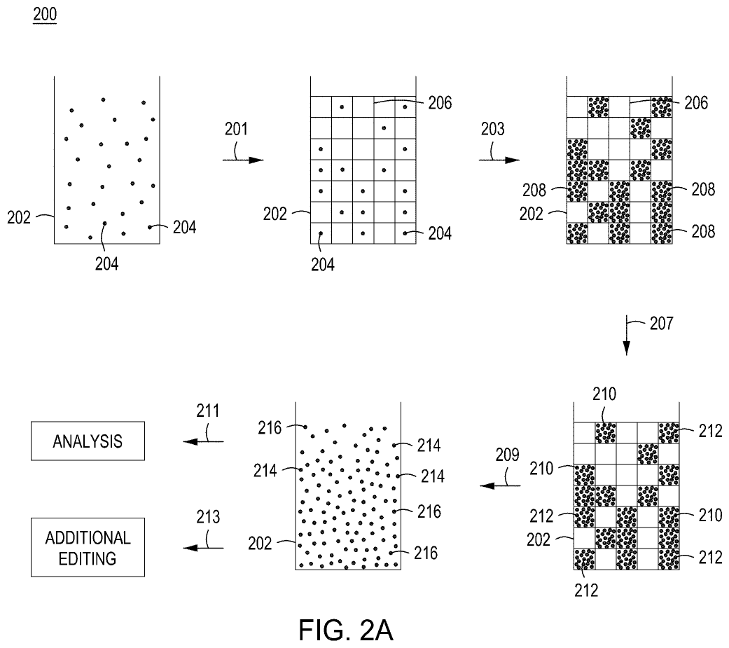

Inducible editing is advantageous in that substantially or largely isolated cells can be grown for several to many cell doublings before editing is initiated, which increases the likelihood that cells with edits will survive, as the double-strand cuts caused by active editing are largely toxic to the cells. This toxicity results both in cell death in the edited colonies, as well as a lag in growth for the edited cells that do survive but must repair and recover following editing. However, once the edited cells have a chance to recover, the size of the colonies of the edited cells will eventually catch up to the size of the colonies of unedited cells (e.g., the process of “normalization” or growing colonies to “terminal size”; see, e.g., FIG. 1B described infra).

In addition to the donor nucleic acid, an editing cassette may comprise one or more primer sites. The primer sites can be used to amplify the editing cassette by using oligonucleotide primers; for example, if the primer sites flank one or more of the other components of the editing cassette.

Also, as described above, the donor nucleic acid may comprise—in addition to the at least one mutation relative to a target sequence—one or more PAM sequence alterations that mutate, delete or render inactive the PAM site in the target sequence. The PAM sequence alteration in the target sequence renders the PAM site “immune” to the nucleic acid-guided nuclease and protects the target sequence from further editing in subsequent rounds of editing if the same nuclease is used.

The editing cassette also may comprise a barcode. A barcode is a unique DNA sequence that corresponds to the donor DNA sequence such that the barcode can identify the edit made to the corresponding target sequence. The barcode can comprise greater than four nucleotides. In some embodiments, the editing cassettes comprise a collection of donor nucleic acids representing, e.g., gene-wide or genome-wide libraries of donor nucleic acids. The library of editing cassettes is cloned into vector backbones where, e.g., each different donor nucleic acid design is associated with a different barcode, or, alternatively, each different cassette molecule is associate with a different barcode.

Additionally, in some embodiments, an expression vector or cassette encoding components of the nucleic acid-guided nuclease system further encodes a nucleic acid-guided nuclease comprising one or more nuclear localization sequences (NLSs), such as about or more than about 1, 2, 3, 4, 5, 6, 7, 8, 9, 10, or more NLSs. In some embodiments, the engineered nuclease comprises NLSs at or near the amino-terminus, NLSs at or near the carboxy-terminus, or a combination.

The engine and editing vectors comprise control sequences operably linked to the component sequences to be transcribed. As stated above, the promoters driving transcription of one or more components of the nucleic acid-guided nuclease editing system may be inducible. A number of gene regulation control systems have been developed for the controlled expression of genes in plant, microbe, and animal cells, including mammalian cells, including the pL promoter (induced by heat inactivation of the CI857 repressor), the pBAD promoter (induced by the addition of arabinose to the cell growth medium), and the rhamnose inducible promoter (induced by the addition of rhamnose to the cell growth medium). Other systems include the tetracycline-controlled transcriptional activation system (Tet-On/Tet-Off, Clontech, Inc. (Palo Alto, Calif.); Bujard and Gossen, PNAS, 89(12):5547-5551 (1992)), the Lac Switch Inducible system (Wyborski et al., Environ Mol Mutagen, 28(4):447-58 (1996); DuCoeur et al., Strategies 5(3):70-72 (1992); U.S. Pat. No. 4,833,080), the ecdysone-inducible gene expression system (No et al., PNAS, 93(8):3346-3351 (1996)), the cumate gene-switch system (Mullick et al., BMC Biotechnology, 6:43 (2006)), and the tamoxifen-inducible gene expression (Zhang et al., Nucleic Acids Research, 24:543-548 (1996)) as well as others. In the present methods used in the modules and instruments described herein, it is preferred that at least one of the nucleic acid-guided nuclease editing components (e.g., the nuclease or the gRNA) is under the control of a promoter that is activated by a rise in temperature, as such a promoter allows for the promoter to be activated by an increase in temperature, and de-activated by a decrease in temperature, thereby “turning off” the editing process. Thus, in the scenario of a promoter that is de-activated by a decrease in temperature, editing in the cell can be turned off without having to change media or addition of other genetic factors in, e.g., an additional genetic element on an existing plasmid, an another plasmid, or integrated into the genome; to remove, e.g., an inducing agent in the medium that is used to induce editing.

FIG. 1A shows simplified flow charts for two alternative exemplary methods 100a and 100b for isolating or substantially isolating cells and normalizing cell colony size where method 100a does not employ induction of the editing machinery and method 100b does employ an inducible promoter driving transcription of the gRNA. Looking at FIG. 1A, method 100a begins by transforming cells 110 with the components necessary to perform nucleic acid-guided nuclease editing. For example, the cells may be transformed simultaneously with separate engine and editing vectors; the cells may already be expressing the nuclease (e.g., the cells may have already been transformed with an engine vector or the coding sequence for the nuclease may be stably integrated into the cellular genome) such that only the editing vector needs to be transformed into the cells; or the cells may be transformed with a single vector comprising all components required to perform nucleic acid-guided nuclease genome editing.

A variety of delivery systems can be used to introduce (e.g., transform or transfect) nucleic acid-guided nuclease editing system components into a host cell 110. These delivery systems include the use of yeast systems, lipofection systems, microinjection systems, biolistic systems, virosomes, liposomes, immunoliposomes, polycations, lipid:nucleic acid conjugates, virions, artificial virions, viral vectors, electroporation, cell permeable peptides, nanoparticles, nanowires, exosomes. Alternatively, molecular trojan horse liposomes may be used to deliver nucleic acid-guided nuclease components across the blood brain barrier. Of interest, particularly in the context of a multi-module cell editing instrument is the use of electroporation, particularly flow-through electroporation (either as a stand-alone instrument or as a module in an automated multi-module system) as described in, e.g., U.S. Ser. No. 16/147,120, filed 28 Sep. 2018; Ser. No. 16/147,353, filed 28 Sep. 2018; Ser. No. 16/147,865, filed 30 Sep. 2018; and Ser. No. 16/426,310, filed 30 May 2019; and U.S. Pat. No. 10,323,258, issued 18 Jun. 2019. If the isolation/growth/editing/normalization module is one module in an automated multi-module cell editing instrument, the cells are likely transformed in an automated cell transformation module.

After the cells are transformed with the components necessary to perform nucleic acid-guided nuclease editing, the cells are isolated or substantially isolated 120; that is, the cells are diluted (if necessary) in a liquid culture medium so that cells are sequestered or separated from one another in the liquid. Isolation can then be performed by solidifying or “gelling” the liquid medium in which the cells are separated from one another in a three-dimensional gel; that is, e.g., the cells are suspended in liquid, then the liquid is solidified into a gel thereby fixing the isolated or singulated cells in three-dimensional space.

Once the cells have been isolated in 100a, the cells are actively editing, as the editing machinery is under the control of a constitutive promoter. As the cells are editing, they are grown into colonies of terminal size 130; that is, the colonies arising from the isolated cells are grown into colonies to a point where cell growth has peaked and is normalized or saturated for both edited and unedited cells. Normalization occurs as the nutrients in the medium around a growing cell colony are depleted. Again, in the embodiment 100a shown in FIG. 1A, the editing components are under the control of a constitutive promoter; thus, editing begins immediately (or almost immediately) upon transformation. However, in other embodiments such as the embodiment shown in 100b, at least the guide nucleic acid (as well as, e.g., λ red recombination system components in bacterial systems) may be under the control of an inducible promoter, in which case editing may be induced after, e.g., a desired number of cell doublings. At this point in method 100a, the terminal-size colonies are pooled 140 by liquefying the solidified medium and, e.g., vortexing the liquid to mix the cells from the normalized colonies. Again, because isolation overcomes growth bias from unedited cells or cells exhibiting fitness effects as the result of edits made, isolation/normalization alone enriches the total population of cells with cells that have been edited; that is, isolation combined with normalization (e.g., growing colonies to terminal size) allows for high-throughput enrichment of edited cells.

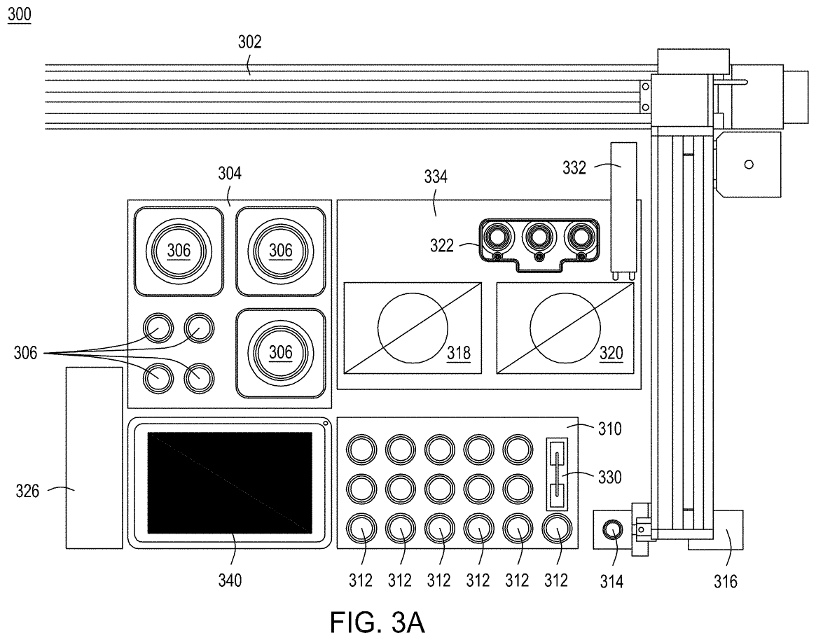

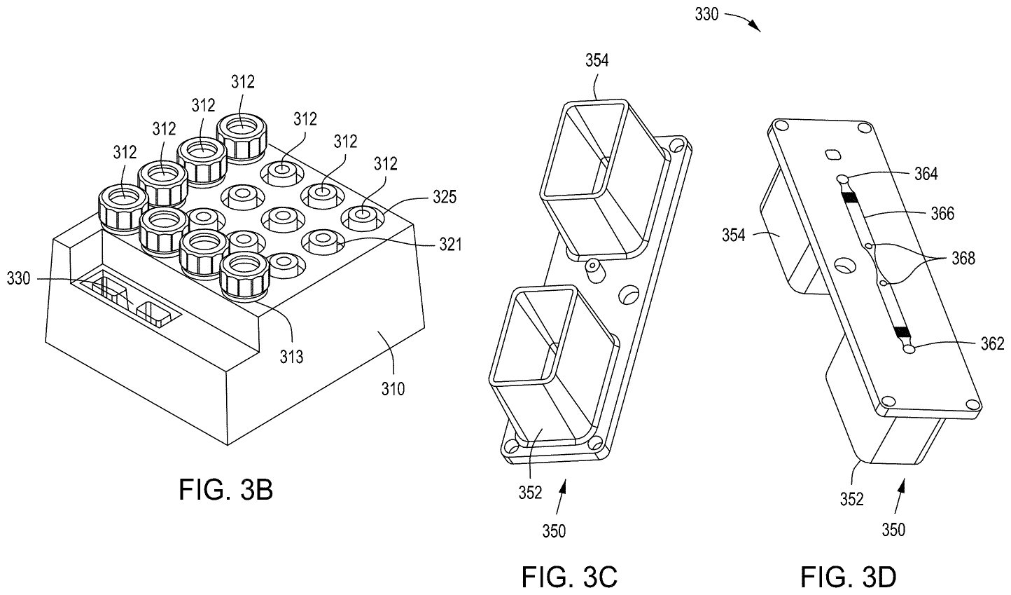

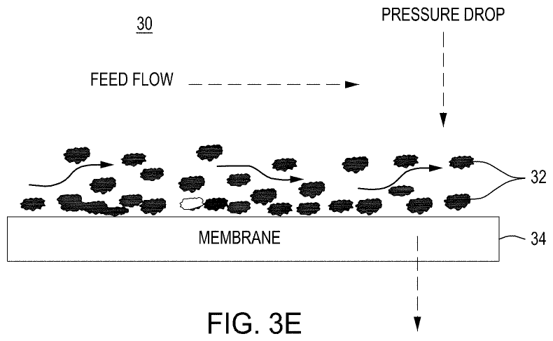

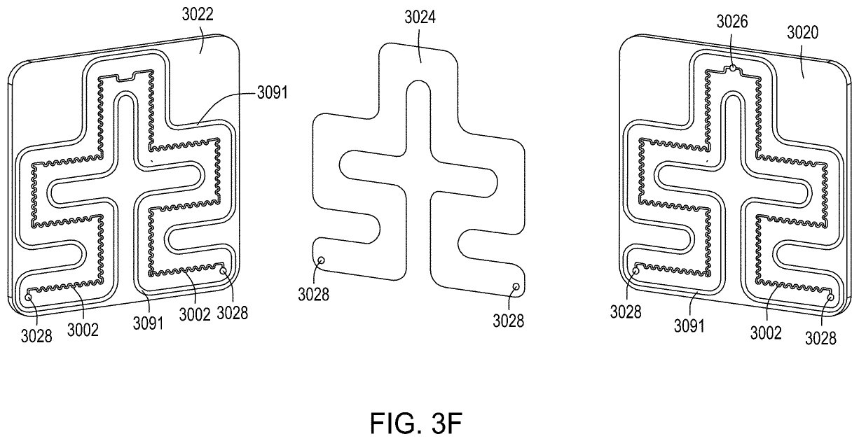

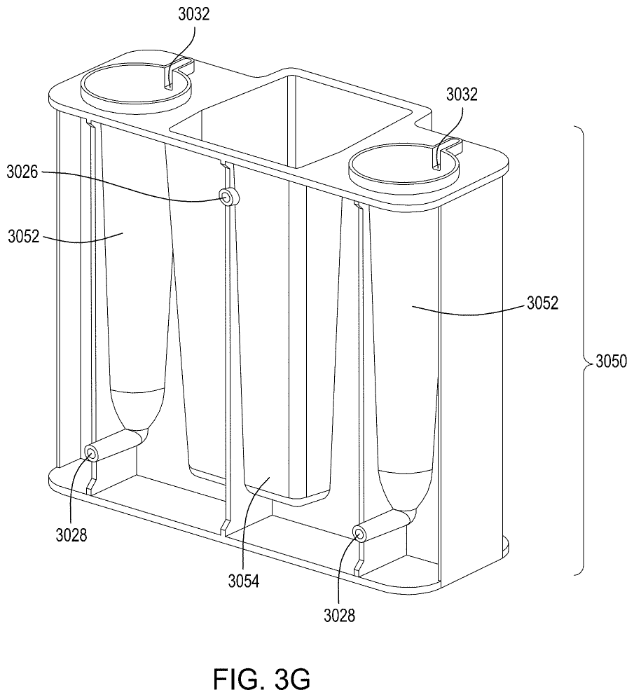

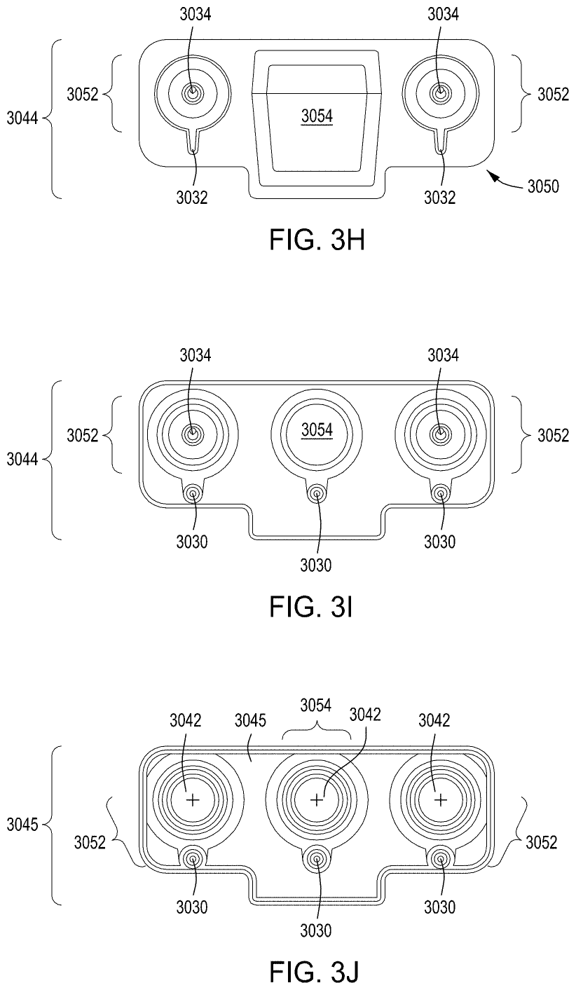

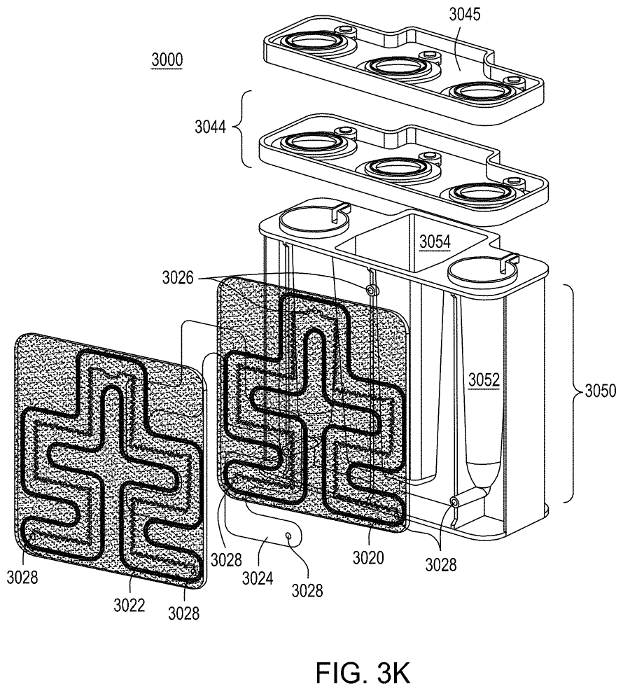

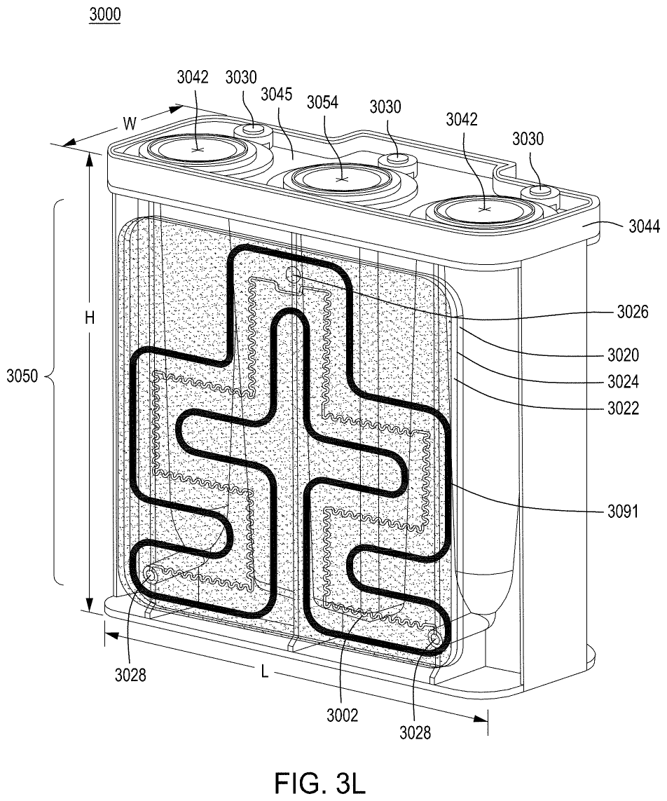

The method 100b shown in FIG. 1A is similar to the method 100a in that cells of interest are transformed 110 with the components necessary to perform nucleic acid-guided nuclease editing. As described above, the cells may be transformed simultaneously with both the engine and editing vectors, the cells may already be expressing the nuclease (e.g., the cells may have already been transformed with an engine vector or the coding sequence for the nuclease may be stably integrated into the cellular genome) such that only the editing vector needs to be transformed into the cells, or the cells may be transformed with a single vector comprising all components required to perform nucleic acid-guided nuclease genome editing. Further, if the isolation/growth/editing/normalization module is one module in an automated multi-module cell editing instrument, cell transformation may be performed in an automated transformation module utilizing a flow-through electroporation module as described in relation to FIGS. 3A-3D below.

After the cells are transformed with the components necessary to perform nucleic acid-guided nuclease editing, the cells are isolated 120; that is, the cells are diluted (if necessary) in liquid medium so that cells are separated from one another. Once the cells are diluted properly in the liquid medium, the liquid medium is solidified or “gelled” fixing and sequestering the diluted cells in three-dimensional space.

Once the cells have been isolated and sequestered 120, the cells are allowed to grow to, e.g., between 2 and 50, or between 5 and 40, or between 10 and 30 doublings, establishing clonal colonies 150. After colonies are established, in this embodiment 100b editing is induced 160 by, e.g., activating inducible promoters that control transcription of one or more of the components needed for nucleic acid-guided nuclease editing, such as, e.g., transcription of the gRNA, donor DNA, nuclease, or, in the case of bacteria, a recombineering system. Once editing is induced 160, many of the edited cells in the clonal colonies die due to the double strand DNA breaks that occur during the editing process; however, in a percentage of the edited cells, the genome is edited, the double strand break is properly repaired, and the cell survives and continues to grow. These edited cells then start growing and re-establish colonies. Once editing is induced and enough time has passed to complete the process, the induced cells are allowed to grow to terminal size 170 (normalized colony size). Then, as in embodiment 100a, the normalized colonies are pooled 140 by liquefying the solidified medium and, e.g., vortexing the liquid to mix the cells from the normalized colonies.

FIG. 1B is a plot of OD versus time for unedited cells (solid line) versus edited cells (dashed line). Note that the OD (e.g., growth) of the edited cells lags behind the unedited cells initially, but eventually catches up due, e.g., to unedited cells exhausting the nutrients in the medium and exiting log-phase growth. The colonies are allowed to grow long enough for the growth of the edited colonies to catch up with (approximate the size of, e.g., number of cells in) the unedited colonies.

### Exemplary Workflows for Editing and Enrichment of Edited Cells

The methods described herein provide enhanced observed editing efficiency of nucleic acid-guided nuclease editing methods as the result of isolation/growth/editing/normalization. The combination of the isolation and normalization processes overcomes the growth bias in favor of unedited cells—and the fitness effects of editing (including differential editing rates)—thus allowing all cells “equal billing” with one another. The result of the methods described herein is that even in nucleic acid-guided nuclease systems where editing is not optimal (such as in systems where non-canonical PAMs are targeted), there is an increase in the observed editing efficiency; that is, edited cells can be identified even in a large background of unedited cells. Observed editing efficiency can be improved up to 80% or more.

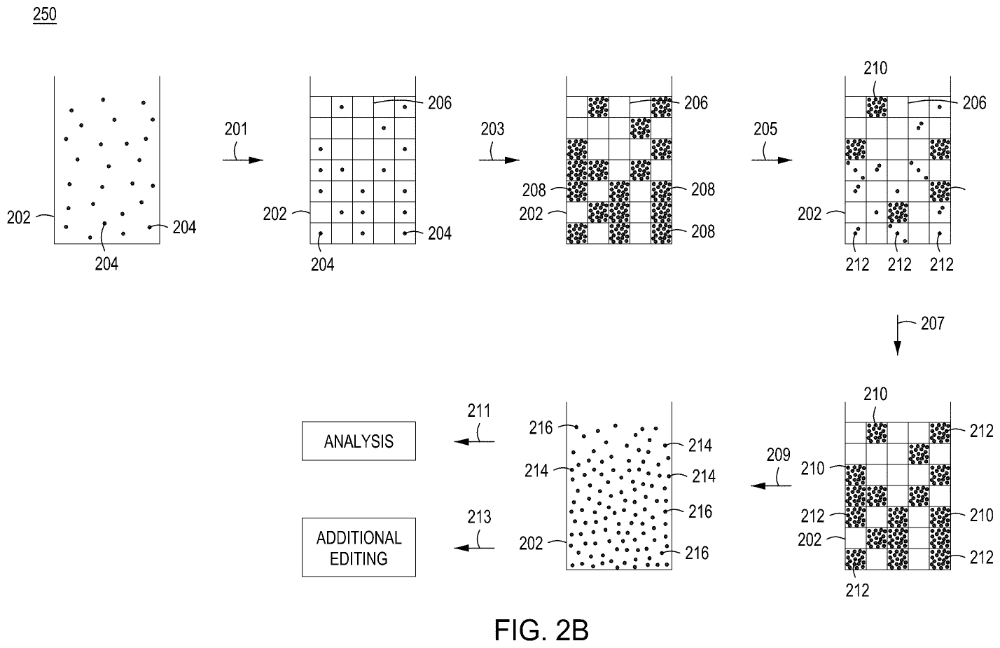

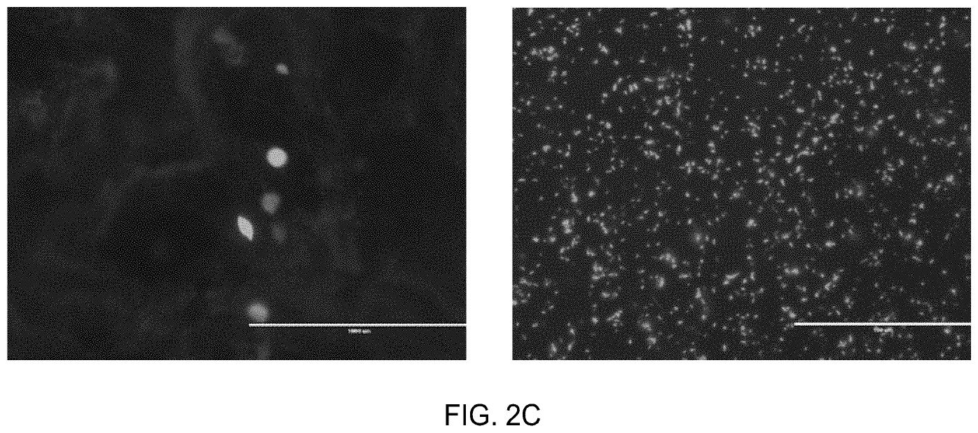

FIG. 2A is an exemplary workflow 200 for optimizing the observed presence of edited cells after nucleic acid-guided nuclease genome editing that may be performed in an automated isolation/growth/editing/normalization module, and, optionally, as part of an automated multi-module cell editing instrument. First, transformed cells 204 are suspended at a pre-determined density in medium plus alginate (solidifying agent) in a vessel 202 containing, optionally, antibiotics or other selective compounds to allow only cells that have been transformed with both the engine vector and editing vector (if two vectors are used) or a combined engine/editing vector to grow. Again, in some embodiments two vectors, an engine vector and an editing vector, are used in some embodiments a single vector comprising all necessary exogenous components for nucleic acid-guided nuclease editing is used. The medium used with depend, of course, on the type of cells being edited—e.g., bacterial, yeast or mammalian. For example, medium for bacterial growth includes LB, SOC, M9 Minimal medium, and Magic medium; medium for yeast cell growth includes TPD, YPG, YPAD, and synthetic minimal medium; and medium for mammalian cell growth includes MEM, DMEM, IMDM, RPMI, and Hanks.

Natural polymers and proteins able to form hydrogels are alginate, chitosan, hyaluronan, dextran, collagen, and fibrin; synthetic examples of synthetic polymers and proteins able to form hydrogels include polyethylene glycol, poly(hydroxyethyl methacrylate, polyvinyl alcohol, and polycaprolactone. Alginate has been used as a preferred solidifying agent in the methods described herein due to a number of advantageous properties. Alginates are polysaccharides that consist of linear (unbranched) 1,4 linked residues of β-D-mannuronic acid and its C5-epimer α-D-guluronic acid. Alginates have a high affinity for alkaline earth metals and ionic hydrogels can be formed in the presence of divalent cations except Mg⁺². Chelation of the gel-forming ion occurs between two consecutive residues in the alginate chain, and an intermolecular gel network is formed as a result of a cooperative binding of consecutive residues in different alginate chains. Advantageously, ionically-gelled alginate can be dissolved by treatment with chelating agents for divalent cations such as citrate and ethylenediaminetetraacetic acid (EDTA) or hexametaphosphate. A 2% (w/v) alginate in medium was found to properly isolate cells; however appropriate ranges for the percentage of alginate in a growth medium include 0.25% to 6% (w/v) alginate, or 0.5% to 5% (w/v) alginate, or 1% to 4% (w/v) alginate, or 2% to 3% (w/v) alginate. In addition, neither of the processes of solidifying and of re-liquefying the alginate/medium (described in more detail below) impact cell viability. Moreover, induction of editing by elevating the temperature of the bulk gel to 42° C. (described in more detail below) does not impact the integrity of the solidified medium or the segregation of the isolated clonal cell colonies.

The culture of mammalian cells using hydrogels has been performed to mimic the 3D cell environments found in tissue, allowing for more biologically-relevant cellular environments. As with tissue mimetics, in the context of mammalian cell editing alginates may be chemically functionalized to alter physiochemical and biological characteristics and properties so as to better bind and promote the growth of mammalian cells once the cells have been isolated in the solidified alginate medium. As cells do not have receptors that recognize alginate, proliferation and differentiation of some mammalian cells within an alginate hydrogen require signaling molecules and matrix interaction. For example, cell attachment peptides, especially the sequence RGD (arginine-glycine-aspartic acid), have been shown to improve cellular adaptability to matricies, as is the case with alginate. Using aqueous carbodiimide chemistry, alginate can be modified by covalently grafting peptide sequence to the alginate molecule. (For a comprehensive discussion of 3D cell culture in alginate hydrogels, see Andersen, et al., Microarrays, 4:133-61 (2015).) Alternatively, as described above, mammalian cells can be grown on beads where the beads are then suspended in the alginate medium.

Once the cells are suspended at an appropriate density, the alginate in the medium is solidified 201 by, e.g., addition of CaCl₂) (described below in relation to Example 5). Note that some areas of the solidified alginate have no cells 206 and some areas have one cell 204. Next, the cells are allowed to grow 203 for a pre-determined approximate number of doublings. Because the cells are fixed in three-dimensional space, the resulting colonies 208 are fixed in three-dimensional space. The colonies are grown to terminal size 207 (that is, edited and non-edited cell colony growth is normalized), sodium citrate is added 209 to the medium such that the solidified medium/alginate re-liquifies and the cells from the colonies 214, 216 (comprising to edited and unedited cells, respectively) are suspended in liquid medium once again. Once the medium is re-liquified, the cells are recovered and subjected to analysis 211 or are used in a second round of editing 213. Again, because the combination of the processes of isolation and normalization overcomes growth bias from unedited cells or cells exhibiting fitness effects as the result of edits made, the combination of the processes of isolation and normalization alone enriches the total population of cells with cells that have been edited; that is, isolation and normalization (e.g., growing colonies to terminal size) allows for high-throughput enrichment of edited cells.

FIG. 2B depicts a simplified graphic for a workflow 250 for isolating, editing and normalizing cells after nucleic acid-guided nuclease genome editing in bulk cell culture, where reversible solidification of the bulk culture is utilized, and editing is induced by inducing transcription of gRNA. First cells 204 are suspended in a vessel at an appropriate density, then at step 201 the alginate in the medium is solidified by, e.g., addition of CaCl₂) (described below in relation to Example 5). Note that some areas of the solidified alginate have no cells 206 and some areas have one cell 204. Next, the cells are allowed to grow 203 for a pre-determined approximate number of doublings. Because the cells are fixed in three-dimensional space, the resulting colonies 208 are fixed in three-dimensional space. Editing is then induced 205 by inducing transcription of the gRNA. Once editing is induced, a number of cells in the edited colonies 212 die due to the toxicity of the double-stranded breaks as the result of editing. Growth of cells in colonies that have not been edited 210 are not affected by double-stranded breaks and continue to thrive. At step 207, the colonies of cells—both edited and unedited—are grown to terminal size (that is, edited 212 and non-edited 210 cell colony growth is normalized), then sodium citrate is added 209 to the medium such that the solidified medium/alginate re-liquifies and the cells from the colonies 214, 216 (comprising to edited and unedited cells, respectively) are suspended in liquid medium once again. Once the medium is re-liquified, the cells are recovered and subjected to analysis 211 or are used in a second round of editing 213. Again, because the combination of the processes of isolation and normalization overcomes growth bias from unedited cells or cells exhibiting fitness effects as the result of edits made, the combination of the processes of isolation and normalization alone enriches the total population of cells with cells that have been edited; that is, isolation and normalization (e.g., growing colonies to terminal size) allows for high-throughput enrichment of edited cells.

FIG. 2C is a photograph of E. coli cells expressing green fluorescent protein in 2.0% alginate and medium that has been solidified showing isolated colonies (left) and a photograph of E. coli cells expressing green fluorescent protein in 2.0% alginate and medium after the medium has been re-liquified.

### Automated Systems Comprising Isolation/Growth/Editing/Normalization Modules

FIG. 3A depicts an exemplary automated multi-module cell processing instrument 300 comprising a bulk cell culture isolation/growth/editing/normalization module 340 to, e.g., perform the exemplary workflows described above in relation to FIGS. 2A and 2B, as well as additional exemplary modules. Illustrated is a gantry 302, providing an automated mechanical motion system (actuator) (not shown) that supplies XYZ axis motion control to, e.g., modules of the automated multi-module cell processing instrument 300, including, e.g., an air displacement pipette 332. In some automated multi-module cell processing instruments, the air displacement pipettor is moved by a gantry and the various modules and reagent cartridges remain stationary; however, in other embodiments, the pipetting system may stay stationary while the various modules are moved. Also included in the automated multi-module cell processing instrument 300 is wash or reagent cartridge 304, comprising reservoirs 306. As described below in respect to FIG. 3B, wash or reagent cartridge 304 may be configured to accommodate large tubes, for example, wash solutions, or solutions that are used often throughout an iterative process. In one example, wash or reagent cartridge 304 may be configured to remain in place when two or more reagent cartridges 310 are sequentially used and replaced. Although reagent cartridge 310 and wash or reagent cartridge 304 are shown in FIG. 3A as separate cartridges, the contents of wash cartridge 304 may be incorporated into reagent cartridge 310.

The exemplary automated multi-module cell processing instrument 300 of FIG. 3A further comprises a cell growth module 334. In the embodiment illustrated in FIG. 3A, the cell growth module 334 comprises two rotating growth vials (RGVs) 318, 320 (described in detail below with relation to FIG. 3E) as well as a cell concentration module 322. In some embodiments, there is a separate isolation/growth/editing and normalization module 340; however, in some embodiments the isolation/growth/editing and normalization module may be a separate RGV in growth module 334, as certain embodiments of the isolation/growth/editing/normalization module utilize a rotating growth vial similar to or the same as the rotating growth vials used to culture the cells before transformation. In addition, once growth, editing and normalization has taken place, cell concentration processes for, e.g., medium exchange and cell concentration may be used to prepare (e.g., concentrate and render electrocompetent) the edited cells for another transformation for another round of editing. Here, the cell concentration module 322 is part of the cell growth module 334; however, in some embodiments the cell concentration module 322 may be separate from cell growth module 334, e.g., in a separate, dedicated module.

Also illustrated as part of the automated multi-module cell processing instrument 300 of FIG. 3A is isolation/growth/editing/normalization module 340 separate from growth module 334, where the module 340 is served by, e.g., air displacement pipettor 332. The isolation/growth/editing/normalization module implementing the workflows described in FIGS. 2A and 2B and illustrated in FIG. 3A may employ “off the shelf” liquid handling instrumentation such as that sold by Opentrons (OT-2™ system, Brooklyn, N.Y.); ThermoFisher Scientific (Versette™ system, Carlsbad, Calif.); Labcyte (Access™ system, San Jose, Calif.); Perkin Elmer (Janus™ system, San Jose, Calif.); Agilent Inc. (Bravo™ system, Santa Clara, Calif.); BioTek Inc. (Winoosky, Vt.); Hudson Inc. (Solo™ system, Springfield, N.J.); Andrew Alliance (Andrew™ system, Waltham, Mass.); and Hamilton Robotics (suite of tools, Reno, Nev.). Also seen in FIG. 3A is a waste repository 326, and a nucleic acid assembly/desalting module 314 comprising a reaction chamber or tube receptacle (not shown) and further comprising a magnet 316 to allow for purification of nucleic acids using, e.g., magnetic solid phase reversible immobilization (SPRI) beads (Applied Biological Materials Inc., Richmond, BC). The reagent cartridge, transformation module, and cell growth module are described in greater detail below.

FIG. 3B depicts an exemplary combination reagent cartridge and electroporation device 310 (“cartridge”) that may be used in an automated multi-module cell processing instrument along with the isolation/growth/editing/normalization module. In certain embodiments the material used to fabricate the cartridge is thermally-conductive, as in certain embodiments the cartridge 310 contacts a thermal device (not shown), such as a Peltier device or thermoelectric cooler, that heats or cools reagents in the reagent receptacles or reservoirs 312. Reagent receptacles or reservoirs 312 may be receptacles into which individual tubes of reagents are inserted as shown in FIG. 3B, or the reagent receptacles may hold the reagents without inserted tubes. Additionally, the receptacles in a reagent cartridge may be configured for any combination of tubes, co-joined tubes, and direct-fill of reagents.

In one embodiment, the reagent receptacles or reservoirs 312 of reagent cartridge 310 are configured to hold various size tubes, including, e.g., 250 ml tubes, 25 ml tubes, 10 ml tubes, 5 ml tubes, and Eppendorf or microcentrifuge tubes. In yet another embodiment, all receptacles may be configured to hold the same size tube, e.g., 5 ml tubes, and reservoir inserts may be used to accommodate smaller tubes in the reagent reservoir (not shown). In yet another embodiment—particularly in an embodiment where the reagent cartridge is disposable—the reagent reservoirs hold reagents without inserted tubes. In this disposable embodiment, the reagent cartridge may be part of a kit, where the reagent cartridge is pre-filled with reagents and the receptacles or reservoirs sealed with, e.g., foil, heat seal acrylic or the like and presented to a consumer where the reagent cartridge can then be used in an automated multi-module cell processing instrument. As one skilled in the art will appreciate given the present disclosure, the reagents contained in the reagent cartridge will vary depending on workflow; that is, the reagents will vary depending on the processes to which the cells are subjected in the automated multi-module cell processing instrument.

Reagents such as cell samples, medium, CaCl₂, Na₃C₆H₅O₇ (sodium citrate), enzymes, buffers, nucleic acid vectors, expression cassettes, proteins or peptides, reaction components (such as, e.g., MgCl₂, dNTPs, nucleic acid assembly reagents, gap repair reagents, and the like), wash solutions, ethanol, and magnetic beads for nucleic acid purification and isolation, etc. may be positioned in the reagent cartridge at a known position. In some embodiments of cartridge 310, the cartridge comprises a script (not shown) readable by a processor (not shown) for dispensing the reagents. Also, the cartridge 310 as one component in an automated multi-module cell processing instrument may comprise a script specifying two, three, four, five, ten or more processes to be performed by the automated multi-module cell processing instrument. In certain embodiments, the reagent cartridge is disposable and is pre-packaged with reagents tailored to performing specific cell processing protocols, e.g., genome editing or protein production. Because the reagent cartridge contents vary while components/modules of the automated multi-module cell processing instrument or system may not, the script associated with a particular reagent cartridge may match the reagents used and cell processes performed. Thus, e.g., reagent cartridges may be pre-packaged with reagents for genome editing and a script that specifies the process steps for performing single or recursive genome editing in an automated multi-module cell processing instrument.

For example, the reagent cartridge may comprise a script to pipette competent cells from a reservoir, transfer the cells to a transformation module (such as flow through electroporation device 330 in reagent cartridge 310), pipette a nucleic acid solution comprising a vector with expression cassette from another reservoir in the reagent cartridge, transfer the nucleic acid solution to the transformation module, initiate the transformation process for a specified time, then move the transformed cells to yet another reservoir in the reagent cassette or to another module such as a cell isolation, editing, and growth module in the automated multi-module cell processing instrument. In another example, the reagent cartridge may comprise a script to transfer a nucleic acid solution comprising a vector from a reservoir in the reagent cassette, nucleic acid solution comprising editing oligonucleotide cassettes in a reservoir in the reagent cassette, and a nucleic acid assembly mix from another reservoir to the nucleic acid assembly/desalting module (314 of FIG. 3A). The script may also specify process steps performed by other modules in the automated multi-module cell processing instrument. For example, the script may specify that the nucleic acid assembly/desalting reservoir be heated to 50° C. for 30 min to generate an assembled product; and desalting and resuspension of the assembled product via magnetic bead-based nucleic acid purification involving a series of pipette transfers and mixing of magnetic beads, ethanol wash, and buffer. These processes are described in greater detail infra.

As described in relation to FIGS. 3C and 3D below, the exemplary reagent cartridges 310 for use in the automated multi-module cell processing instruments may include one or more electroporation devices 330, preferably flow-through electroporation devices. Electroporation is a widely-used method for permeabilization of cell membranes that works by temporarily generating pores in the cell membranes with electrical stimulation. Applications of electroporation include the delivery of DNA, RNA, siRNA, peptides, proteins, antibodies, drugs or other substances to a variety of cells such as mammalian cells (including human cells), plant cells, archea, yeasts, other eukaryotic cells, bacteria, and other cell types. Electrical stimulation may also be used for cell fusion in the production of hybridomas or other fused cells. During a typical electroporation procedure, cells are suspended in a buffer or medium that is favorable for cell survival. For bacterial cell electroporation, low conductance mediums, such as water, glycerol solutions and the like, are often used to reduce the heat production by transient high current. In traditional electroporation devices, the cells and material to be electroporated into the cells (collectively “the cell sample”) are placed in a cuvette embedded with two flat electrodes for electrical discharge. For example, Bio-Rad (Hercules, Calif.) makes the GENE PULSER XCELL™ line of products to electroporate cells in cuvettes. Traditionally, electroporation requires high field strength; however, the flow-through electroporation devices included in the reagent cartridges such as those shown in FIGS. 3B-3D achieve high efficiency cell electroporation with low toxicity. The reagent cartridges of the disclosure allow for particularly easy integration with robotic liquid handling instrumentation that is typically used in automated instruments and systems such as air displacement pipettors. Such automated instrumentation includes, but is not limited to, off-the-shelf automated liquid handling systems from Tecan (Mannedorf, Switzerland), Hamilton (Reno, Nev.), Beckman Coulter (Fort Collins, Colo.), etc. as described above.

FIGS. 3C and 3D are top perspective and bottom perspective views, respectively, of an exemplary flow-through electroporation device 350 that may be part of reagent cartridge 300 in FIG. 3B or may be contained in a separate module (e.g., a transformation/transfection module). FIG. 3C depicts a flow-through electroporation unit 350. The flow-through electroporation unit 350 has wells that define cell sample inlets 352 and cell sample outlets 354. FIG. 3D is a bottom perspective view of the flow-through electroporation device 350 of FIG. 3C. An inlet well 352 and an outlet well 354 can be seen in this view. Also seen in FIG. 3D are the bottom of an inlet 362 corresponding to well 352, the bottom of an outlet 364 corresponding to the outlet well 354, the bottom of a defined flow channel 366 and the bottom of two electrodes 368 on either side of flow channel 366. Additionally, flow-through electroporation devices may comprise push-pull pneumatic means to allow multi-pass electroporation procedures; that is, cells to be electroporated may be “pulled” from the inlet toward the outlet for one pass of electroporation, then be “pushed” from the outlet end of the flow-through electroporation device toward the inlet end to pass between the electrodes again for another pass of electroporation. Exemplary flow-through electroporation devices of use in the automated multi-module cell processing instruments disclosed herein include those described in U.S. Ser. No. 16/147,120, filed 28 Sep. 2018; Ser. No. 16/147,353, filed 28 Sep. 2018; Ser. No. 16/147,865, filed 30 Sep. 2018; and Ser. No. 16/426,310, filed 30 May 2019; and U.S. Pat. No. 10,323,258, issued 18 Jun. 2019, all of which are herein incorporated by reference in their entirety. Further, this process may be repeated one to many times. Moreover, other embodiments of the reagent cartridge may provide or accommodate electroporation devices that are not configured as flow-through devices, such as those described in U.S. Ser. No. 16/109,156, filed 22 Aug. 2018.

Exemplary automated multi-module cell processing instrument 300 of FIG. 3A also comprises a nucleic acid assembly module. The nucleic acid assembly module 314 is configured to perform, e.g., an isothermal nucleic acid assembly. An isothermal nucleic acid assembly joins multiple DNA fragments in a single, isothermal reaction, requiring few components and process manipulations. For example, an isothermal nucleic acid assembly can combine simultaneously up to 20 or more nucleic acid fragments based on sequence identity. The assembly method requires that the nucleic acids to be assembled comprise at least a 15-base overlap with adjacent nucleic acid fragments. The fragments are mixed with a cocktail of three enzymes—an exonuclease, a polymerase, and a ligase—along with buffer components. Because in some embodiments the process is isothermal and can be performed in a 1-step or 2-step method using a single reaction vessel, the isothermal nucleic acid assembly method is suited for use in an automated multi-module cell processing instrument. The 1-step method allows for the assembly of up to five different fragments using a single step isothermal process. The fragments and the master mix of enzymes are combined and incubated at 50° C. for up to one hour. For the creation of more complex constructs or for incorporating fragments from 100 bp up to 10 kb, typically the 2-step method is used, where the 2-step reaction requires two separate additions of master mix; one for the exonuclease and annealing step and a second for the polymerase and ligation steps.

In an embodiment of the exemplary automated multi-module cell processing instrument 300 of FIG. 3A, aliquots of a vector, an oligonucleotide (e.g., a gene of interest or an editing sequence) to be inserted into the vector, and the nucleic acid assembly mix may be retrieved from three of the sixteen reagent reservoirs 312 disposed within reagent cartridge 310. The vector, oligonucleotide, and reaction mix are combined in a reaction chamber or tube located in a tube receptacle (not shown) in the nucleic acid assembly module, and the module is heated to 50° C. After the nucleic acid assembly reaction has taken place, magnetic beads may be retrieved from one of the reagent reservoirs 312 disposed within reagent cartridge 310 and added to the nucleic acid assembly mix in the reaction chamber of the nucleic acid module 314. As seen in FIG. 3A, magnet 316, such as a solenoid magnet, is adjacent or proximal to the nucleic acid assembly module 314. Once the magnetic beads are added to the nucleic acid assembly reaction the nucleic acid product binds the magnetic beads, and after a period of incubation magnet 316 is engaged, isolating the magnetic beads coupled to the nucleic acids in the reaction chamber. The reaction solution (supernatant) in the nucleic acid assembly module 314 can be removed by air displacement pipettor 332, and a wash solution and/or ethanol may be pipetted from a reagent reservoir 312 in reagent cartridge 310, or from a wash solution reservoir 306 in wash cartridge 304 and used to wash the nucleic acids coupled to the beads. The magnet may be disengaged while the beads and coupled nucleic acids are being washed, then the magnet would be re-engaged to remove the wash solution from the nucleic acid assembly module. Alternatively, the magnet may not be disengaged while the beads and coupled nucleic acids are washed. The de-salted assembled vector+oligo may then be moved to, e.g., the flow-through electroporation device (transformation/transfection module) as described in relation to FIGS. 3B through 3D.

FIG. 3E is a model of tangential flow filtration used in the TFF module described below. The TFF device is an integral module in the automated multi-module cell processing instrument. The TFF is used to concentrate and render electrocompetent cells after growth in the cell growth module. The cells may be cells that were loaded into a rotating growth vial for a first round of editing, or the cells may be cells that have been through one round of editing, recovered from liquefied alginate medium, re-grown in a rotating growth vial and are being prepared for a second round of editing. The TFF device was designed to take into account two primary design considerations. First, the geometry of the TFF device leads to filtering of the cell culture over a large surface area so as to minimize processing time. Second, the design of the TFF device is configured to minimize filter fouling. FIG. 3E is a general model 30 of tangential flow filtration. The TFF device operates using tangential flow filtration, also known as cross-flow filtration. FIG. 3E shows cells flowing over a membrane 34, where the feed flow of the cells 32 in medium or buffer is parallel to the membrane 34. TFF is different from dead-end filtration where both the feed flow and the pressure drop are perpendicular to a membrane or filter.

FIGS. 3F-7L depict an embodiment of a tangential flow filtration (TFF) device/module. FIG. 3F depicts a configuration of an retentate member 3022 (on left), a membrane or filter 3024 (middle), and a permeate member 3020 (on the right). In FIG. 3F, retentate member 3022 comprises a tangential flow channel 3002, which has a serpentine configuration that initiates at one lower corner of retentate member 3022—specifically at retentate port 3028—traverses across and up then down and across retentate member 3022, ending in the other lower corner of retentate member 3022 at a second retentate port 3028. Also seen on retentate member 3022 is energy director 3091, which circumscribes the region where membrane or filter 3024 is seated. Energy director 3091 in this embodiment mates with and serves to facilitate ultrasonic wending or bonding of retentate member 3022 with permeate member 3020 via the energy director component on permeate member 3020. Membrane or filter 3024 has through-holes for retentate ports 3028 and is configured to seat within the circumference of energy directors 3091 between the retentate member 3022 and permeate member 3020. Permeate member 3020 comprises, in addition to energy director 3091, through-holes for retentate port 3028 at each bottom corner (which mate with the through-holes for retentate ports 3028 at the bottom corners of membrane 3024 and retentate ports 3028 in retentate member 3022), as well as a tangential flow channel 3002 and a single permeate port 3026 positioned at the top and center of permeate member 3020. The tangential flow channel 3002 structure in this embodiment has a serpentine configuration and an undulating geometry, although other geometries may be used. In some aspects, the length of the tangential flow channel is from 10 mm to 1000 mm, from 60 mm to 200 mm, or from 80 mm to 100 mm. In some aspects, the width of the channel structure is from 10 mm to 120 mm, from 40 mm to 70 mm, or from 50 mm to 60 mm. In some aspects, the cross section of the tangential flow channel 1202 is rectangular. In some aspects, the cross section of the tangential flow channel 1202 is 5 μm to 1000 μm wide and 5 μm to 1000 μm high, 300 μm to 700 μm wide and 300 μm to 700 μm high, or 400 μm to 600 μm wide and 400 μm to 600 μm high. In other aspects, the cross section of the tangential flow channel 1202 is circular, elliptical, trapezoidal, or oblong, and is 100 μm to 1000 μm in hydraulic radius, 300 μm to 700 μm in hydraulic radius, or 400 μm to 600 μm in hydraulic radius.

FIG. 3G is a side perspective view of a reservoir assembly 3050. Reservoir assembly 3050 comprises retentate reservoirs 3052 on either side of a single permeate reservoir 3054. Retentate reservoirs 3052 are used to contain the cells and medium as the cells are transferred through the TFF device or module and into the retentate reservoirs during cell concentration. Permeate reservoir 3054 is used to collect the filtrate fluids removed from the cell culture during cell concentration, or old buffer or medium during cell growth. In the embodiment depicted in FIGS. 3F-3L, buffer or medium is supplied to the permeate member from a reagent reservoir separate from the device module. Additionally seen in FIG. 3G are grooves 3032 to accommodate pneumatic ports (not seen), permeate port 3026, and retentate port through-holes 3028. The retentate reservoirs are fluidically coupled to the retentate ports 3028, which in turn are fluidically coupled to the portion of the tangential flow channel disposed in the retentate member (not shown). The permeate reservoir is fluidically coupled to the permeate port 3026 which in turn are fluidically coupled to the portion of the tangential flow channel disposed in permeate member (not shown), where the portions of the tangential flow channels are bifurcated by membrane (not shown). In embodiments including the present embodiment, up to 120 mL of cell culture can be grown and/or filtered, or up to 100 mL, 90 mL, 80 mL, 70 mL, 60 mL, 50 mL, 40 mL, 30 mL or 20 mL of cell culture can be grown and/or concentrated.

FIG. 3H depicts a top-down view of the reservoir assembly 3050 shown in FIG. 3G, FIG. 3I depicts a cover 3044 for reservoir assembly 3050 shown in FIGS. 3G, and 3J depicts a gasket 3045 that in operation is disposed on cover 3044 of reservoir assembly 3050 shown in FIG. 3G. FIG. 3H is a top-down view of reservoir assembly 3050, showing two retentate reservoirs 3052, one on either side of permeate reservoir 3054. Also seen are grooves 3032 that will mate with a pneumatic port (not shown), and fluid channels 3034 that reside at the bottom of retentate reservoirs 3052, which fluidically couple the retentate reservoirs 3052 with the retentate ports 3028 (not shown), via the through-holes for the retentate ports in permeate member 3220 and membrane 3024 (also not shown). FIG. 3I depicts a cover 3044 that is configured to be disposed upon the top of reservoir assembly 3050. Cover 3044 has round cut-outs at the top of retentate reservoirs 3052 and permeate reservoir 3054. Again, at the bottom of retentate reservoirs 3052 fluid channels 3034 can be seen, where fluid channels 3034 fluidically couple retentate reservoirs 3052 with the retentate ports 3028 (not shown). Also shown are three pneumatic ports 3030 for each retentate reservoir 3052 and permeate reservoir 3054. FIG. 3J depicts a gasket 3045 that is configured to be disposed upon the cover 3044 of reservoir assembly 3050. Seen are three fluid transfer ports 3042 for each retentate reservoir 3052 and for permeate reservoir 3054. Again, three pneumatic ports 3030, for each retentate reservoir 3052 and for permeate reservoir 3054, are shown.

FIG. 3K depicts an exploded view of a TFF module 3000. Seen are components reservoir assembly 3050, a cover 3044 to be disposed on reservoir assembly 3050, a gasket 3045 to be disposed on cover 3044, retentate member 3022, membrane or filter 3024, and permeate member 3020. Also seen is permeate port 3026, which mates with permeate port 3026 on permeate reservoir 3054, as well as two retentate ports 3028, which mate with retentate ports 3028 on retentate reservoirs 3052 (where only one retentate reservoir 3052 can be seen clearly in this FIG. 3K). Also seen are through-holes for retentate ports 3028 in membrane 3024 and permeate member 3020.

FIG. 3L depicts an embodiment of assembled TFF module 3000. Retentate member 3022, membrane member 3024, and permeate member 3020 are coupled side-to-side with reservoir assembly 3050. Seen are two retentate ports 3028 (which couple the tangential flow channel 3002 in retentate member 3022 to the two retentate reservoirs (not shown), and one permeate port 3026, which couples the tangential flow channel 3002 in permeate/filtrate member 3020 to the permeate reservoir (not shown). Also seen is tangential flow channel 3002, which is formed by the mating of retentate member 3022 and permeate member 3020, with membrane 3024 sandwiched between and bifurcating tangential flow channel 3002. Also seen is energy director 3091, which in this FIG. 3L has been used to ultrasonically weld or couple retentate member 3022 and permeate member 3020, surrounding membrane 3024. Cover 3044 can be seen on top of reservoir assembly 3050, and gasket 3045 is disposed upon cover 3044. Gasket 3045 engages with and provides a fluid-tight seal and pneumatic connections with fluid transfer ports 3042 and pneumatic ports 3030, respectively. FIG. 3L also shows the length, height, and width dimensions of the TFF module 3000. The assembled TFF device 3000 typically is from 50 to 175 mm in height, or from 75 to 150 mm in height, or from 90 to 120 mm in height; from 50 to 175 mm in length, or from 75 to 150 mm in length, or from 90 to 120 mm in length; and is from 30 to 90 mm in depth, or from 40 to 75 mm in depth, or from about 50 to 60 mm in depth. An exemplary TFF device is 110 mm in height, 120 mm in length, and 55 mm in depth. For further information and alternative embodiments on TFFs, see, e.g., U.S. Ser. No. 62/728,365, filed 7 Sep. 2018; 62/857,599, filed 5 Jun. 2019; and 62/867,415, filed 27 Jun. 2019.

### Use of a Growth Module in the Automated Instrument to Perform Bulk Gel Culture

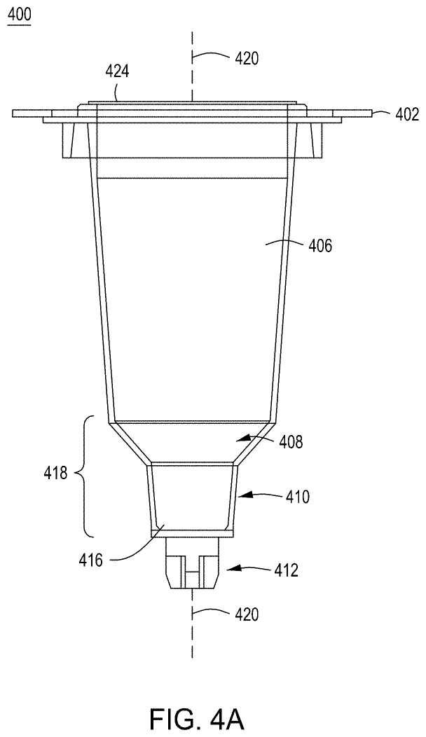

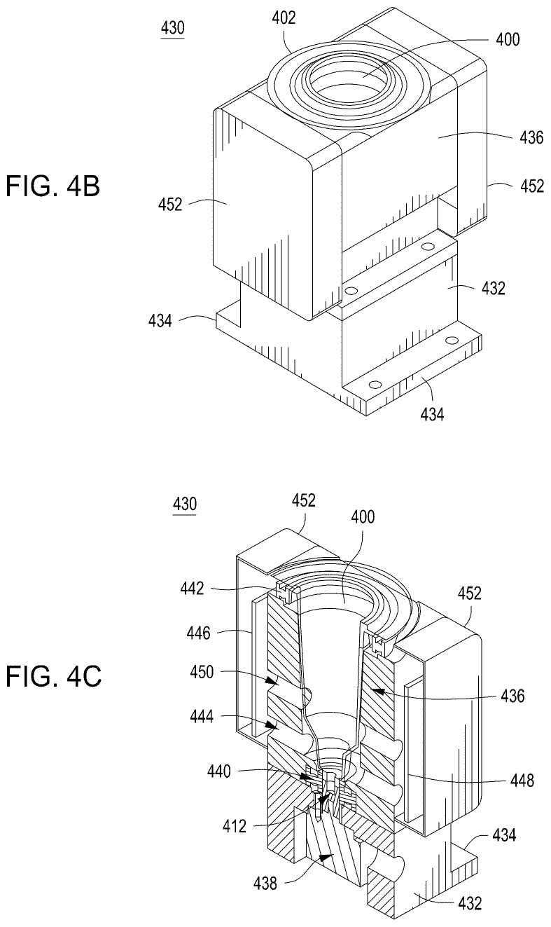

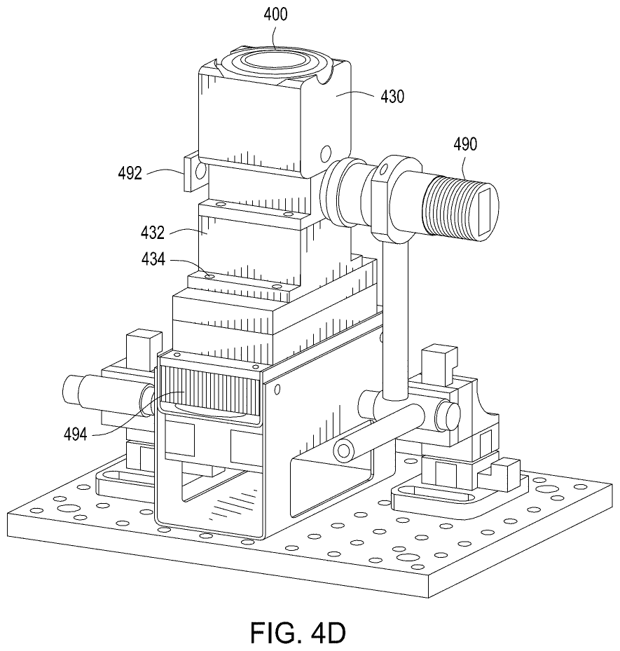

FIG. 4A depicts one embodiment of a rotating growth vial (RGV) that may be used 1) to grow cells to an appropriate OD for transformation, and 2) as a vessel for the bulk cell culture procedures depicted in FIGS. 1A, 2A and 2B. In an embodiment where the RGV is used to grow cells for transformation, the RGV may constantly measure the optical density of a growing cell culture. One advantage of the cell growth module is that optical density can be measured continuously (kinetic monitoring) or at specific time intervals; e.g., every 5, 10, 15, 20, 30 45, or 60 seconds, or every 1, 2, 3, 4, 5, 6, 7, 8, 9, or 10 or so on minutes. Alternatively, OD can be measured at specific time intervals early in the cell growth cycle, and continuously after the OD of the cell culture reaches a set point OD. The cell growth module is controlled by a processor, which can be programmed to measure OD constantly or at intervals as defined by a user. A script on, e.g., the reagent cartridge(s) may also specify the frequency for reading OD, as well as the target OD and target time. Additionally, a user manually can set a target time at which the user desires the cell culture hit a target OD. To accomplish reaching the target OD at the target time, the processor measures the OD of the growing cells, calculates the cell growth rate in real time, and predicts the time the target OD will be reached. The processor then automatically adjusts the temperature of the RGV (and the cell culture) as needed. Lower temperatures slow growth, and higher temperatures increase growth.

In the RGV embodiment depicted in FIG. 4A, the RGV 400 is a transparent container having an open end 424 for receiving liquid media and cells, a central vial region 406 that defines the primary container for growing cells, a tapered-to-constricted region 418 defining at least one light path 410, a closed end 416, and a drive engagement mechanism 412. The RGV has a central longitudinal axis 420 around which the vial rotates, and the light path 410 is generally perpendicular to the longitudinal axis of the vial. The first light path 410 is positioned in the lower constricted portion of the tapered-to-constricted region 408. Optionally, some embodiments of the RGV 400 have a second light path 418 in the tapered region of the tapered-to-constricted region 408. Both light paths in this embodiment are positioned in a region of the RGV that is constantly filled with the cell culture (cells+growth media) and are not affected by the rotational speed of the RGV. The first light path 410 is shorter than the second light path 418 allowing for sensitive measurement of OD values when the OD values of the cell culture in the vial are at a high level (e.g., later in the cell growth process), whereas the second light path 418 allows for sensitive measurement of OD values when the OD values of the cell culture in the vial are at a lower level (e.g., earlier in the cell growth process). The drive engagement mechanism 412 engages with a motor (not shown) to rotate the vial.

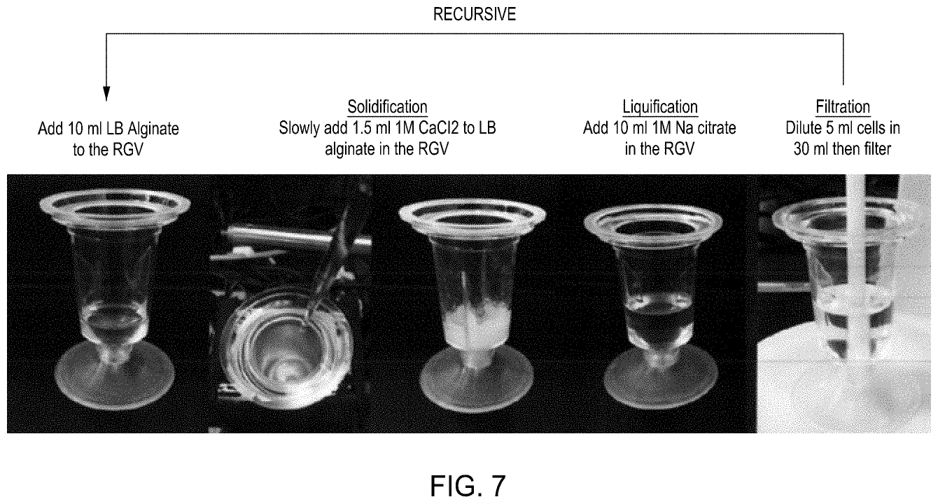

The RGV 400 may be reusable, or preferably, the RGV—like the reagent cartridge—is consumable. In some embodiments, the RGV is consumable and is presented to a user pre-filled with growth medium, where the vial is sealed at the open end 424 with a foil seal. The growth module comprising the rotating growth vial depicted in FIGS. 4A-4D may also be employed as the vessel for bulk cell culture, isolation, editing, and pooling (see, e.g., FIG. 7).

When the rotating growth vial RGV) is used as an isolation module, cells in medium containing 0.25%-6% alginate are transferred into the rotating growth vial by, e.g., a liquid handling system, where first, the cells are at an appropriate dilution to allow each cell to be isolated or substantially isolated from other cells when the medium is gelled, and second, the cell colonies that grow from the isolated cells in the gelled or solidified medium are isolated from other cell colonies. Once the cells at the proper dilution are loaded into the RGV, solidification or gelling of the medium is triggered by slowing adding an appropriate amount of, e.g., CaCl₂ dropwise to the RGV, preferably while the RGV is spinning at a low speed. Once the medium is solidifies, the cells can be grown to colonies of terminal size (e.g., normalized) (see, e.g., FIG. 2A, where no induction of editing takes place) or the cells can be grown for, e.g., 2-50 doublings, editing is then induced by, e.g., raising the temperature of the RGV to 42° C. for a period of time to induce a pL promoter driving transcription of the gRNA, then the temperature is lowered and the cells are allowed to grow to terminal size or a desired concentration of cells (see, e.g., FIG. 2B). After the cells have grown to terminal size (e.g., the cells are in senescence and the cell colonies are in no longer increasing in size), the gelled or solidified medium is liquefied by adding an appropriate amount of, e.g., sodium citrate to the solidified medium dropwise preferably while the RGV is spinning at a low speed. The cells and medium may then be removed from the RGV by the liquid handling system and filtered in, e.g., a filtration module such as the FTT device as described in relation to FIGS. 3F-3L.

FIG. 4B is a perspective view of one embodiment of a cell growth device 430. FIG. 4C depicts a cut-away view of the cell growth device 430 from FIG. 4B. In both figures, the rotating growth vial 400 is seen positioned inside a main housing 436 with the extended lip 402 of the rotating growth vial 400 extending above the main housing 436. Additionally, end housings 452, a lower housing 432 and flanges 434 are indicated in both figures. Flanges 434 are used to attach the cell growth device 430 to heating/cooling means or other structure (not shown). FIG. 4C depicts additional detail. In FIG. 4C, upper bearing 442 and lower bearing 440 are shown positioned within main housing 636. Upper bearing 442 and lower bearing 440 support the vertical load of rotating growth vial 400. Lower housing 432 contains the drive motor 438. The cell growth device 430 of FIG. 4C comprises two light paths: a primary light path 444, and a secondary light path 450. Light path 444 corresponds to light path 410 positioned in the constricted portion of the tapered-to-constricted portion of the rotating growth vial 400, and light path 450 corresponds to light path 408 in the tapered portion of the tapered-to-constricted portion of the rotating growth vial 400. Light paths 410 and 408 are not shown in FIG. 4C but may be seen in FIG. 4A. In addition to light paths 444 and 440, there is an emission board 448 to illuminate the light path(s), and detector board 446 to detect the light after the light travels through the cell culture liquid in the rotating growth vial 400.

The motor 438 engages with drive mechanism 412 and is used to rotate the rotating growth vial 400. In some embodiments, motor 438 is a brushless DC type drive motor with built-in drive controls that can be set to hold a constant revolution per minute (RPM) between 0 and about 3000 RPM. Alternatively, other motor types such as a stepper, servo, brushed DC, and the like can be used. Optionally, the motor 438 may also have direction control to allow reversing of the rotational direction, and a tachometer to sense and report actual RPM. The motor is controlled by a processor (not shown) according to, e.g., standard protocols programmed into the processor and/or user input, and the motor may be configured to vary RPM to cause axial precession of the cell culture thereby enhancing mixing, e.g., to prevent cell aggregation, increase aeration, and optimize cellular respiration.

Main housing 436, end housings 452 and lower housing 432 of the cell growth device 430 may be fabricated from any suitable, robust material including aluminum, stainless steel, and other thermally conductive materials, including plastics. These structures or portions thereof can be created through various techniques, e.g., metal fabrication, injection molding, creation of structural layers that are fused, etc. Whereas the rotating growth vial 400 is envisioned in some embodiments to be reusable, but preferably is consumable, the other components of the cell growth device 430 are preferably reusable and function as a stand-alone benchtop device or as a module in a multi-module cell processing system.

The processor (not shown) of the cell growth device 430 may be programmed with information to be used as a “blank” or control for the growing cell culture. A “blank” or control is a vessel containing cell growth medium only, which yields 100% transmittance and 0 OD, while the cell sample will deflect light rays and will have a lower percent transmittance and higher OD. As the cells grow in the media and become denser, transmittance will decrease and OD will increase. The processor (not shown) of the cell growth device 430—may be programmed to use wavelength values for blanks commensurate with the growth media typically used in cell culture (whether, e.g., mammalian cells, bacterial cells, animal cells, yeast cells, etc.). Alternatively, a second spectrophotometer and vessel may be included in the cell growth device 430, where the second spectrophotometer is used to read a blank at designated intervals.

FIG. 4D illustrates a cell growth device 430 as part of an assembly comprising the cell growth device 430 of FIG. 4B coupled to light source 490, detector 492, and thermal components 494. The rotating growth vial 400 is inserted into the cell growth device. Components of the light source 490 and detector 492 (e.g., such as a photodiode with gain control to cover 5-log) are coupled to the main housing of the cell growth device. The lower housing 432 that houses the motor that rotates the rotating growth vial 400 is illustrated, as is one of the flanges 434 that secures the cell growth device 430 to the assembly. Also, the thermal components 494 illustrated are a Peltier device or thermoelectric cooler. In this embodiment, thermal control is accomplished by attachment and electrical integration of the cell growth device 430 to the thermal components 494 via the flange 434 on the base of the lower housing 432. Thermoelectric coolers are capable of “pumping” heat to either side of a junction, either cooling a surface or heating a surface depending on the direction of current flow. In one embodiment, a thermistor is used to measure the temperature of the main housing and then, through a standard electronic proportional-integral-derivative (PID) controller loop, the rotating growth vial 400 is controlled to approximately +/−0.5° C.

In use, cells are inoculated (cells can be pipetted, e.g., from an automated liquid handling system or by a user) into pre-filled growth media of a rotating growth vial 400 by piercing though the foil seal or film. The programmed software of the cell growth device 430 sets the control temperature for growth, typically 30° C., then slowly starts the rotation of the rotating growth vial 400. The cell/growth media mixture slowly moves vertically up the wall due to centrifugal force allowing the rotating growth vial 400 to expose a large surface area of the mixture to a normal oxygen environment. The growth monitoring system takes either continuous readings of the OD or OD measurements at pre-set or pre-programmed time intervals. These measurements are stored in internal memory and if requested the software plots the measurements versus time to display a growth curve. If enhanced mixing is required, e.g., to optimize growth conditions, the speed of the vial rotation can be varied to cause an axial precession of the liquid, and/or a complete directional change can be performed at programmed intervals. The growth monitoring can be programmed to automatically terminate the growth stage at a pre-determined OD, and then quickly cool the mixture to a lower temperature to inhibit further growth.

One application for the cell growth device 430 is to constantly measure the optical density of a growing cell culture. One advantage of the described cell growth device is that optical density can be measured continuously (kinetic monitoring) or at specific time intervals; e.g., every 5, 10, 15, 20, 30 45, or 60 seconds, or every 1, 2, 3, 4, 5, 6, 7, 8, 9, or 10 minutes. While the cell growth device 430 has been described in the context of measuring the optical density (OD) of a growing cell culture, it should, however, be understood by a skilled artisan given the teachings of the present specification that other cell growth parameters can be measured in addition to or instead of cell culture OD. As with optional measure of cell growth in relation to the solid wall device or module described supra, spectroscopy using visible, UV, or near infrared (NIR) light allows monitoring the concentration of nutrients and/or wastes in the cell culture and other spectroscopic measurements may be made; that is, other spectral properties can be measured via, e.g., dielectric impedance spectroscopy, visible fluorescence, fluorescence polarization, or luminescence. Additionally, the cell growth device 630 may include additional sensors for measuring, e.g., dissolved oxygen, carbon dioxide, pH, conductivity, and the like.

### Methods for Using the Instrument to Edit Cells in Bulk Gel

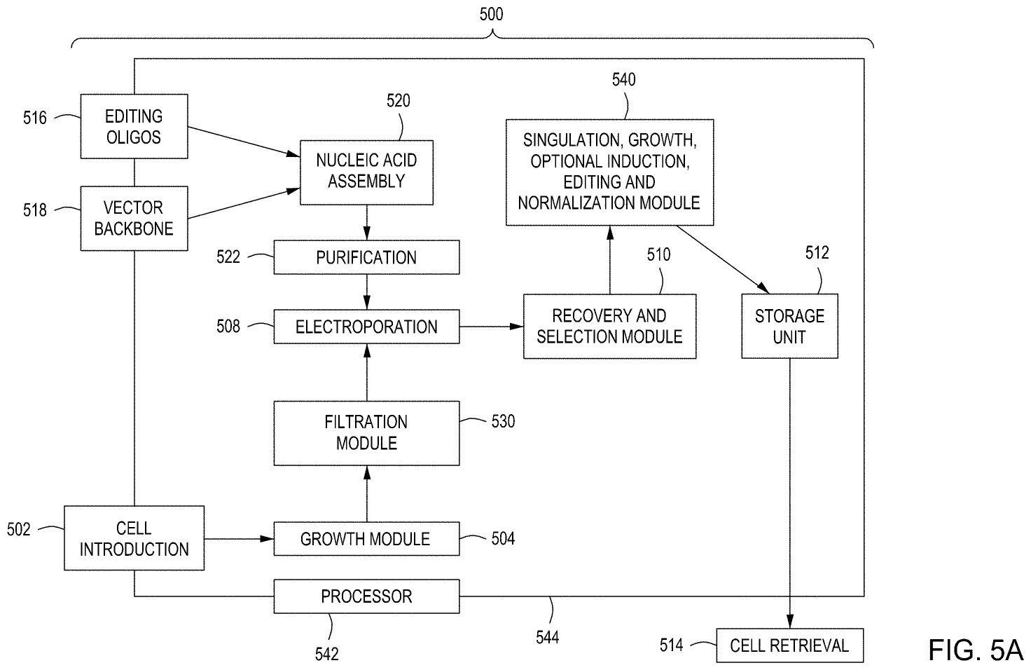

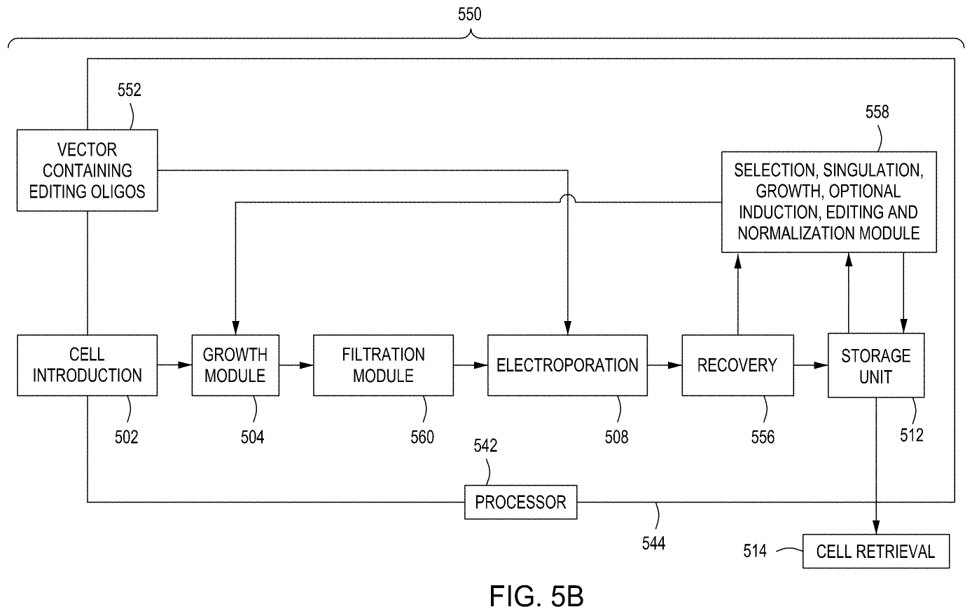

FIG. 5A is a simplified block diagram of an embodiment of an exemplary automated multi-module cell processing instrument comprising an isolation/growth/editing/normalization module for enrichment for edited cells. The cell processing instrument 500 may include a housing 544, a reservoir of cells to be transformed or transfected 502, and a growth module (a cell growth device) 504. The cells to be transformed are transferred from a reservoir to the growth module to be cultured until the cells hit a target OD. Once the cells hit the target OD, the growth module may cool or freeze the cells for later processing, or the cells may be transferred to an optional filtration module 530 where the cells are rendered electrocompetent and concentrated to a volume optimal for cell transformation. Once concentrated, the cells are then transferred to the electroporation device 608 (e.g., transformation/transfection module).

In addition to the reservoir for storing the cells, the system 500 may include a reservoir for storing editing oligonucleotide cassettes 516 and a reservoir for storing an expression vector backbone 518. Both the editing oligonucleotide cassettes and the expression vector backbone are transferred from the reagent cartridge to a nucleic acid assembly module 520, where the editing oligonucleotide cassettes are inserted into the expression vector backbone. The assembled nucleic acids may be transferred into an optional purification module 522 for desalting and/or other purification and/or concentration procedures needed to prepare the assembled nucleic acids for transformation. Alternatively, pre-assembled nucleic acids, e.g., an editing vector, may be stored within reservoir 516 or 518. Once the processes carried out by the purification module 522 are complete, the assembled nucleic acids are transferred to, e.g., an electroporation device 508, which already contains the cell culture grown to a target OD and rendered electrocompetent via filtration module 530. In electroporation device 508, the assembled nucleic acids are introduced into the cells. Following electroporation, the cells are transferred into a combined recovery/selection module 510. For examples of multi-module cell editing instruments, see U.S. Pat. No. 10,253,316, issued 9 Apr. 2019; U.S. Pat. No. 10,329,559, issued 25 Jun. 2019; U.S. Pat. No. 10,323,242, issued 18 Jun. 2019; and U.S. Ser. No. 16/412,175, filed 14 May 2019; Ser. No. 16/412,195, filed 14 Jun. 2019; and Ser. No. 16/423,289, filed 29 May 2019, all of which are herein incorporated by reference in their entirety.

Following recovery and optionally, selection, the cells are transferred to an isolation, editing, and growth module 540, where the cells are diluted and compartmentalized such that there is an average of one cell per compartment. Once isolated, the cells are allowed to grow to terminal size (e.g., the colonies are normalized). Once the colonies are grown to terminal size, the colonies are pooled. Again, isolation overcomes growth bias from unedited cells and growth bias resulting from fitness effects of different edits.

The recovery, selection, isolation, editing and growth modules may all be separate, may be arranged and combined as shown in FIG. 5A, or may be arranged or combined in other configurations. In certain embodiments, such as those described in relation to the rotating growth vial shown in FIG. 4A, all of recovery, selection, isolation, editing, and normalization are performed in a single vessel/module (e.g., a rotating growth vial 400 in growth module 430 of FIG. 4B).

Once the normalized cell colonies are pooled, the cells may be stored, e.g., in a storage module 512, where the cells can be kept at, e.g., 4° C. until the cells are retrieved for further study. Alternatively, the cells may be used in another round of editing. The multi-module cell processing instrument is controlled by a processor 542 configured to operate the instrument based on user input, as directed by one or more scripts, or as a combination of user input or a script. The processor 542 may control the timing, duration, temperature, and operations of the various modules of the system 600 and the dispensing of reagents. For example, the processor 542 may cool the cells post-transformation until editing is desired, upon which time the temperature may be raised to a temperature conducive of genome editing and cell growth. The processor may be programmed with standard protocol parameters from which a user may select, a user may specify one or more parameters manually or one or more scripts associated with the reagent cartridge may specify one or more operations and/or reaction parameters. In addition, the processor may notify the user (e.g., via an application to a smart phone or other device) that the cells have reached the target OD as well as update the user as to the progress of the cells in the various modules in the multi-module system.

The automated multi-module cell processing instrument 500 is a nuclease-directed genome editing system and can be used in single editing systems (e.g., introducing one or more edits to a cellular genome in a single editing process). The system of FIG. 5B, described below, is configured to perform sequential editing, e.g., using different nuclease-directed systems sequentially to provide two or more genome edits in a cell; and/or recursive editing, e.g. utilizing a single nuclease-directed system to introduce sequentially two or more genome edits in a cell.

FIG. 5B illustrates another embodiment of a multi-module cell processing instrument. This embodiment depicts an exemplary system that performs recursive gene editing on a cell population. As with the embodiment shown in FIG. 5A, the cell processing instrument 550 may include a housing 544, a reservoir for storing cells to be transformed or transfected 502, and a cell growth module comprising a rotating growth vial 504. The cells to be transformed are transferred from a reservoir to the cell growth module to be cultured until the cells hit a target OD. Once the cells hit the target OD, the growth module may cool or freeze the cells for later processing or transfer the cells to an a TFF module 530 where the cells are subjected to buffer exchange and rendered electrocompetent, and the volume of the cells may be reduced substantially. Once the cells have been concentrated to an appropriate volume, the cells are transferred to electroporation device 508. In addition to the reservoir for storing cells, the multi-module cell processing instrument includes a reservoir for storing the vector pre-assembled with editing oligonucleotide cassettes 506. The pre-assembled nucleic acid vectors are transferred to the electroporation device 508, which already contains the cell culture grown to a target OD. In the electroporation device 508, the nucleic acids are electroporated into the cells. Following electroporation, the cells are transferred into an optional recovery module 556, where the cells are allowed to recover briefly post-transformation.

After recovery, the cells may be transferred to a storage module 512, where the cells can be stored at, e.g., 4° C. for later processing, or the cells may be transferred to a selection/isolation/editing/normalization module 558. In the isolation/edit/growth module 558, the cells are diluted such that cells are isolated from one another in three-dimensional space. The arrayed cells are in selection medium to select for cells that have been transferred or transfected with the editing vectors. Once isolated, the liquid medium is solidified, and the cells continue to grow to form clonal colonies in three-dimensional space. Optionally, editing is induced by providing conditions (e.g., temperature, addition of an inducing or repressing chemical) to induce editing. Once editing proceeds, the cells are allowed to grow to terminal size or desired cell concentration or optical density (e.g., normalization of the colonies) and then are pooled and transferred to the storage unit 514 or can be transferred to a growth module 504 for another round of growth, transformation and editing. In between pooling and transfer to a growth module, there may be one or more additional steps, such as medium exchange, cell concentration, etc., by, e.g., filtration via a TFF. Note that the selection/isolation/growth/editing and normalization modules may be the same module, where all processes are performed in the same vessel such as the rotating growth vial of FIGS. 4A-4D. Once the putatively-edited cells are pooled, they may be subjected to another round of editing, beginning with growth, cell concentration and treatment to render electrocompetent, and transformation by yet another donor nucleic acid in another editing cassette via the electroporation module 508.

In electroporation device 508, the cells from the first round of editing are transformed by a second set of editing oligos (or other type of oligos) and the cycle is repeated until the cells have been transformed and edited by a desired number of, e.g., editing cassettes. The multi-module cell processing instrument exemplified in FIG. 5B is controlled by a processor 542 configured to operate the instrument based on user input or is controlled by one or more scripts including at least one script associated with the reagent cartridge. The processor 542 may control the timing, duration, and temperature of various processes, the dispensing of reagents, and other operations of the various modules of the system 550. For example, a script or the processor may control the dispensing of cells, reagents, vectors, and editing oligonucleotides; which editing oligonucleotides are used for cell editing and in what order; the time, temperature and other conditions used in the recovery and expression module, the wavelength at which OD is read in the cell growth module, the target OD to which the cells are grown, and the target time at which the cells will reach the target OD. In addition, the processor may be programmed to notify a user (e.g., via an application) as to the progress of the cells in the automated multi-module cell processing instrument.

### EXAMPLES

The following examples are put forth so as to provide those of ordinary skill in the art with a complete disclosure and description of how to make and use the present invention, and are not intended to limit the scope of what the inventors regard as their invention nor are they intended to represent that the experiments below are all or the only experiments performed. Other equivalent methods, steps and compositions are intended to be included in the scope of the invention. Efforts have been made to ensure accuracy with respect to numbers used (e.g. amounts, temperature, etc.) but some experimental errors and deviations should be accounted for. Unless indicated otherwise, parts are parts by weight, molecular weight is weight average molecular weight, temperature is in degrees Celsius, and pressure is at or near atmospheric.

### Example 1: Editing Cassette and Backbone Amplification and Assembly

Editing Cassette Preparation:

5 nM oligonucleotides synthesized on a chip were amplified using Q5 polymerase in 50 μL volumes. The PCR conditions were 95° C. for 1 minute; 8 rounds of 95° C. for 30 seconds/60° C. for 30 seconds/72° C. for 2.5 minutes; with a final hold at 72° C. for 5 minutes. Following amplification, the PCR products were subjected to SPRI bead cleanup, where 30 μL SPRI mix was added to the 50 μL PCR reactions and incubated for 2 minutes. The tubes were subjected to a magnetic field for 2 minutes, the liquid was removed, and the beads were washed 2× with 80% ethanol, allowing 1 minute between washes. After the final wash, the beads were allowed to dry for 2 minutes, 50 μL 0.5×TE pH 8.0 was added to the tubes, and the beads were vortexed to mix. The slurry was incubated at room temperature for 2 minutes, then subjected to the magnetic field for 2 minutes. The eluate was removed and the DNA quantified.

Following quantification, a second amplification procedure was carried out using a dilution of the eluate from the SPRI cleanup. PCR was performed under the following conditions: 95° C. for 1 minute; 18 rounds of 95° C. for 30 seconds/72° C. for 2.5 minutes; with a final hold at 72° C. for 5 minutes. Amplicons were checked on a 2% agarose gel and pools with the cleanest output(s) were identified. Amplification products appearing to have heterodimers or chimeras were not used.

Backbone Preparation:

A 10-fold serial dilution series of purified backbone was performed, and each of the diluted backbone series was amplified under the following conditions: 95° C. for 1 minute; then 30 rounds of 95° C. for 30 seconds/60° C. for 1.5 minutes/72° C. for 2.5 minutes; with a final hold at 72° C. for 5 minutes. After amplification, the amplified backbone was subjected to SPRI cleanup as described above in relation to the cassettes. The backbone was eluted into 100 μL ddH₂O and quantified before nucleic acid assembly.

Isothermal Nucleic Acid Assembly:

150 ng backbone DNA was combined with 100 ng cassette DNA. An equal volume of 2× Gibson Master Mix was added, and the reaction was incubated for 45 minutes at 50° C. After assembly, the assembled backbone and cassettes were subjected to SPRI cleanup, as described above.

### Example 2: Creation of Cell Line Transformed with Engine Vector

Transformation:

1 μL of the engine vector DNA (comprising a coding sequence for MAD7 nuclease under the control of the pL inducible promoter, a chloramphenicol resistance gene, and the λ Red recombineering system) was added to 50 μL EC1 strain E. coli cells. The transformed cells were plated on LB plates with 25 μg/mL chloramphenicol (chlor) and incubated overnight to accumulate clonal isolates. The next day, a colony was picked, grown overnight in LB+25 μg/mL chlor, and glycerol stocks were prepared from the saturated overnight culture by adding 500 μL 50% glycerol to 1000 μL culture. The stocks of EC1 comprising the engine vector were frozen at −80° C.

### Example 3: Preparation of Competent Cells

A 1 mL aliquot of a freshly-grown overnight culture of EC1 cells transformed with the engine vector was added to a 250 mL flask containing 100 mL LB/SOB+25 μg/mL chlor medium. The cells were grown to 0.4-0.7 OD, and cell growth was halted by transferring the culture to ice for 10 minutes. The cells were pelleted at 8000×g in a JA-18 rotor for 5 minutes, washed 3× with 50 mL ice cold ddH₂0 or 10% glycerol and pelleted at 8000×g in JA-18 rotor for 5 minutes. The washed cells were resuspended in 5 mL ice cold 10% glycerol and aliquoted into 200 μL portions. Optionally at this point the glycerol stocks could be stored at −80° C. for later use.

### Example 4: Bulk Cell 3D Isolation, Colony Normalization, and Processing within a Rotating Growth Vial

Editing Bulk Cell Culture:

This protocol describes a standard bulk culture protocol using alginate as the solidifying agent. Alginate is solidified by addition of CaCl2 and liquefies upon the addition of a chelating agent, and both processes can take place at a temperature appropriate for enriching for nucleic acid-guided nuclease editing of bacterial and yeast cells by isolation, growth, editing, and normalization. This protocol was used to leverage the inducible system for both the nuclease and gRNAs to allow for a phenotypic difference in colonies. Alginate (Alginate, A1112 Sigma-Aldrich (St. Louis, Mo.), Alginic acid sodium salt from brown algae, low viscosity).

### Solutions:

TABLE 1
LB Alginate

| | 1 L | 500 ml | 250 ml | units | notes |
|---|---|---|---|---|---|
| LB | 25 | 12.5 | 6.25 | g | LB Miller version of LB |
| powder | | | | | Broth powder (Teknova |
| | | | | | Cat. No. L9135) |
| DI H₂O | 1000 | 500 | 250 | ml | |
| Alginate | 20 | 10 | 5 | g | Alginic acid sodium salt |
| | | | | | from brown algae, low |
| | | | | | viscosity (A1112 Sigma); |
| | | | | | 2% final conc |

LB and DI H₂O in desired quantities as listed in Table 1 were combined in a flask. A stir bar was added to the flask and the alginate was added slowly while the LB/alginate mixture was stirred on a stir plate. The LB/alginate mixture was then sterilized by autoclavation using standard conditions (e.g., 121° C., 20 min, liquid cycle). After autoclavation, the solution was immediately cooled on ice. Before using the LB/alginate solution, cells and desired antibiotics were added to the appropriate concentration.

TABLE 2
LB Alginate, composition for arabinose induction (1% final conc)

| | 1 L | 500 ml | 250 ml | units | notes |
|---|---|---|---|---|---|
| LB | 25 | 12.5 | 6.25 | g | LB Miller version of LB |
| powder | | | | | Broth powder (Teknova |
| | | | | | Cat. No. L9135) |
| DI H₂O | 950 | 475 | 237.5 | ml | |
| alginate | 20 | 10 | 5 | g | Alginic acid sodium salt |
| | | | | | from brown algae, low |
| | | | | | viscosity (A1112 Sigma); |
| | | | | | 2% final conc |
| 20% | 50 | 25 | 12.5 | ml | From 20% Arabinose |
| arabi- | | | | | Solution, 1 Liter, |
| nose | | | | | Sterile. (Teknova Cat. |
| | | | | | No. A2100); 1% final |
| | | | | | conc, to be added after |
| | | | | | autoclavation, just before use |

LB and DI H₂O in desired quantities as listed in Table 2 were combined in a bottle. A stir bar was added to the bottle and the alginate was added slowly while the LB/alginate mixture was stirred on a stir plate. The LB/alginate mixture was then sterilized by autoclavation using standard conditions (e.g., 121° C., 20 min, liquid cycle). After autoclavation, the solution was immediately cooled on ice. Before using the LB/alginate solution, cells and desired antibiotics were added to the appropriate concentration, and 1 ml of 20% arabinose also was added to 19 ml of the LB alginate solution to obtain a 1% arabinose final concentration. Next, calcium chloride (1M) solution was prepared, using calcium chloride dihydrate, MW=147.01 g/mol, and this calcium chloride solution was filter sterilized. Also, a 1M sodium citrate solution was prepared, using sodium citrate tribasic dihydrate, MW=294.10 g/mol, which was also filter sterilized.

Editing was performed following the above protocols to make LB Alginate (25 ml per sample) and LB Alginate+1% arabinose (25 ml per sample). 10 ml of alginate+1% arabinose solution was added to each 50 ml conical tube. The conical tubes were kept at 30° C., to be ready for use after transformation protocol was complete. Transformation was performed using 500 ng of the nucleic acid assembly (vector+editing cassette library) into ec83 (recombineering competent cells) using the Nepagene electroporator settings for E. coli. The cells were allowed to recover in 3000 μl of SOB in 15 ml conical tubes while shaking at 30° C. for 3 hours. After 3 hours, the alginate tubes and the transformation tubes were removed from the 30° C. incubator, and 250 μl of cells was added to each tube with the 25 ml of Alginate solution (1:10). The Alginate solution was solidified by slowly transferring 20 ml of the alginate+cells solution into 30 ml of 100 mM CaCl₂) solution. The alginate slurry was then centrifuged for 10 min at 4000×g. The supernatant was decanted, and the bulk gel was incubated at 30° C. for 9 hours. After the 9-hour 30° C. incubation, the temperature was shifted to 42° C. for 2 hours for induction of editing.

After editing, the temperature was shifted back to 30° C. for growth overnight. To re-liquify (dissolve) the alginate, 40 ml DI water was added to each conical tube, and 10 ml of 1M sodium citrate was added. The tubes were then shaken at 30° C. for 30-45 minutes. For singleplex recovery, the libraries were recovered by diluting the cells and plating the cells on selective media plates. Various dilutions were plated and plates also were spotted to get colony counts. The cells were grown on the selective medium for 12-24 hours, and colonies were picked into a 96-well plate with each well containing 750 ml LB. The picked colonies were grown for 24 hours, and each sample was prepped for DNA extraction and next-gen sequencing. For amplicon recovery, the cells were spun at 5,000×g for 10 minutes. The supernatant was removed and the cells were resuspended in 500 μl of 0.8 NaCl. A Zyppy™ Plasmid Miniprep kit (Zymo Research, Bath, UK) was used to extract the plasmid DNA from the library, and the samples were prepped for PCR of the inserts, and for assaying the amplicons via next-gen sequencing.

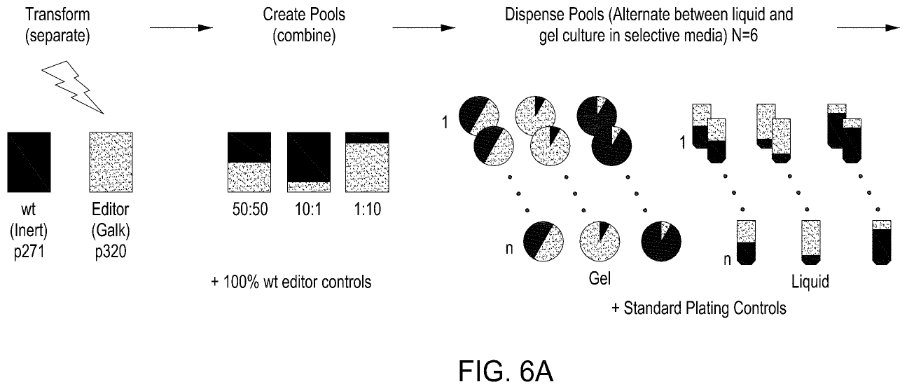

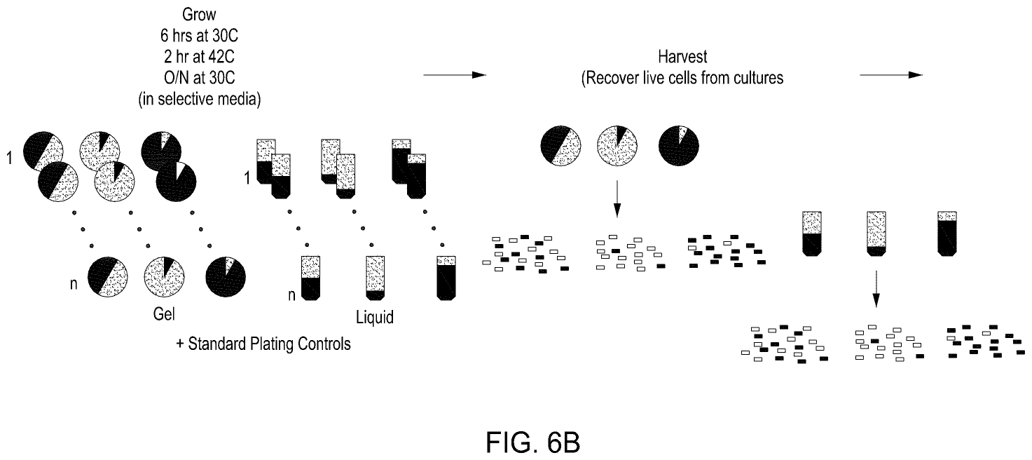

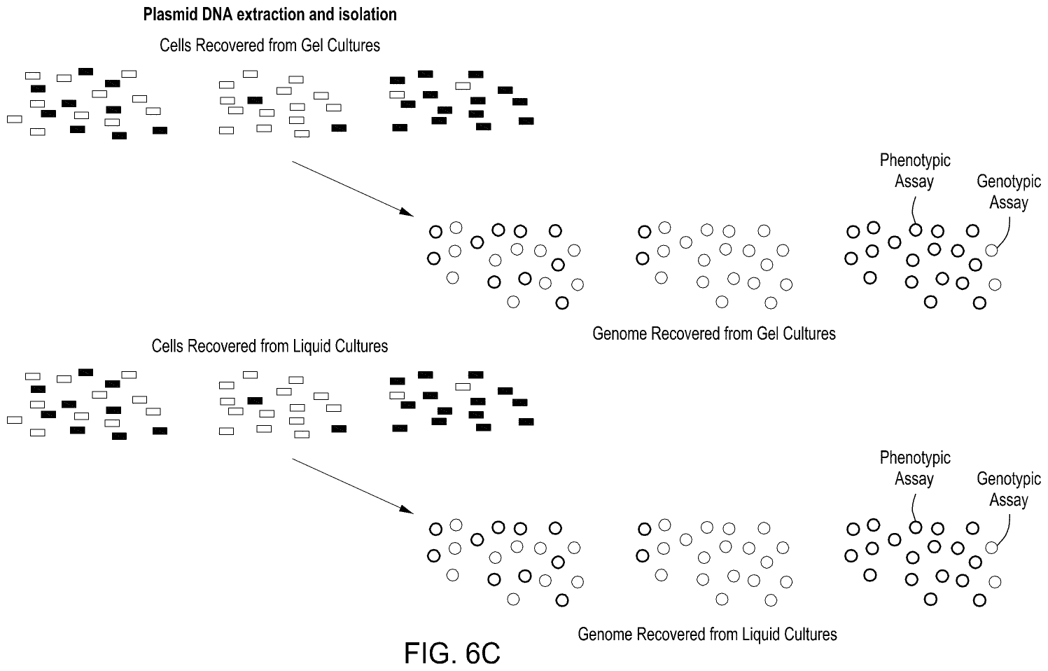

FIGS. 6A-6C is a depiction of an experiment performed to demonstrate that normalization is achieved in bulk culture, which compares the quantity of wildtype (inert) plasmid and editing plasmid (GalK) in bulk gel versus liquid cell culture (see Example 7 below). In a first step (shown in FIG. 6A), the wildtype plasmid was used to transform an E. coli cell line, and separately, an editing plasmid was used to transform the E. coli cell line. Once transformed, pools of the transformed cells were combined in the following ratios: 50:50, 10:1, and 1:10 (wildtype to editing cells, respectively) and dispensed between both bulk culture and liquid culture, where six replicates were prepared for each. Controls included 100% wildtype and 100% editing cells, and standard plating controls. In FIG. 6B, the bulk and liquid cultures (experimental and controls) were grown at 30° C. for 6 hours, 42° C. for 2 hours, and at 30° C. overnight. Next, live cells were recovered from each culture (e.g., six experimental cultures and controls for each of the bulk and liquid cultures). FIG. 6C depicts plasmid extraction and isolation of the cells recovered from the bulk gel cultures and from the liquid cultures (shown) as well as the controls (not shown). Phenotypic assessment was used to determine whether normalization takes place in the bulk gel culture. The phenotypic read out comprised red/white screening on MacConkey agar. The results obtained demonstrated cells edited in bulk gel match most closely the loaded ratio of the 50:50 mix of cells edited at 25% in alginate and 7% liquid and on a plate.

FIG. 7 depicts the workflow for bulk alginate isolation, growth, induction, editing, and normalization in a module comprising a rotating growth vial (as shown in FIG. 4E and described above), which can be used in a multi-module cell editing system (as shown in FIG. 3A and described above). In a first step, 10 ml LB medium comprising alginate was added to the rotating growth vial, which already contained the transformed cells to be edited. In addition to the alginate, the medium also comprises antibiotics to select for the cells that have been properly transformed. The medium was then solidified by slowly adding 1.5 ml of 1M CaCL₂ to the LB alginate cell culture in the rotating growth vial. The cells were allowed to grow for 6 hours at 30° C. to establish cell colonies, 2 hours at 42° C. (which induces editing), then overnight at 30° C. to normalize the edited and unedited cell colonies. After normalization, the solidified LB alginate medium was liquified by adding 10 ml 1 M sodium citrate to the solidified medium, and the liquified normalized cell culture was filtered in a filtration module allowing for buffer exchange, cell concentration, and, if desired, rendering the cells electrocompetent for an additional round of editing. Liquification disperses all cells throughout the culture.

It should be apparent to one of ordinary skill in the art given the present disclosure that the process described may be recursive; that is, cells may go through the workflow described in relation to FIG. 7, then the resulting edited culture may go through another (or several to many) rounds of additional editing (e.g., recursive editing) with different editing vectors. For example, the cells from round 1 of editing may be diluted and an aliquot of the edited cells edited by editing vector A may be combined with editing vector B, an aliquot of the edited cells edited by editing vector A may be combined with editing vector C, an aliquot of the edited cells edited by editing vector A may be combined with editing vector D, and so on for a second round of editing. After round two, an aliquot of each of the double-edited cells may be subjected to a third round of editing, where, e.g., aliquots of each of the AB-, AC-, AD-edited cells are combined with additional editing vectors, such as editing vectors X, Y, and Z. That is that double-edited cells AB may be combined with and edited by vectors X, Y, and Z to produce triple-edited edited cells ABX, ABY, and ABZ; double-edited cells AC may be combined with and edited by vectors X, Y, and Z to produce triple-edited cells ACX, ACY, and ACZ; and double-edited cells AD may be combined with and edited by vectors X, Y, and Z to produce triple-edited cells ADX, ADY, and ADZ, and so on. In this process, many permutations and combinations of edits can be executed, leading to very diverse cell populations and cell libraries. In any recursive process, it is advantageous to “cure” the previous engine and editing vectors (or single engine+editing vector in a single vector system). “Curing” is a process in which one or more vectors used in the prior round of editing is eliminated from the transformed cells. Curing can be accomplished by, e.g., cleaving the vector(s) using a curing plasmid thereby rendering the editing and/or engine vector (or single, combined vector) nonfunctional; diluting the vector(s) in the cell population via cell growth (that is, the more growth cycles the cells go through, the fewer daughter cells will retain the editing or engine vector(s)), or by, e.g., utilizing a heat-sensitive origin of replication on the editing or engine vector (or combined vector). The conditions for curing will depend on the mechanism used for curing; that is, in this example, how the curing plasmid cleaves the editing and/or engine plasmid. Curing in the context of the isolation, growth, editing, and normalization reactions described herein is described in Example 8.

### Example 5: Standard Plating for Comparison to Bulk Culture

This protocol describes a standard plating protocol for enriching for nucleic acid-guided nuclease editing of bacterial cells by isolation, growth, editing, and normalization. This protocol was used to leverage the inducible system for both the nuclease and gRNAs to allow for a phenotypic difference in colonies. From the resulting agar plates, it is possible to select edited cells with a high degree (˜80%) of confidence. Though clearly this protocol can be employed for enriching for edited cells, in the experiments described herein this “standard plating protocol” of “SPP” was used to compare efficiencies of isolation, editing, and normalization with the bulk cell culture. The protocols for liquid cell culture described in Example 7 were used for the same purpose.

Materials: Outside of standard molecular biology tools, the following will be necessary:

TABLE 3

| Product | Vendor |
|---|---|
| SOB | Teknova |
| LB | Teknova |
| LB agar plate with | Teknova |
| chloramphenicol/carbenicillin | |
| and 1% arabinose | |

Protocol: Inputs for this protocol are frozen electrocompetent cells and purified nucleic acid assembly product. Immediately after electroporation, the cell/DNA mixture was transferred to a culture tube containing 2.7 mL of SOB medium. Preparing 2.7 mL aliquots in 14 mL culture tubes prior to electroporation allowed for a faster recovery of cells from the electroporation cuvette; the final volume of the recovery was 3 mL. All culture tubes were placed into a shaking incubator set to 250 RPM and 30° C. for three hours. While the cultures were recovering, the necessary number of LB agar plates with chloramphenicol and carbenicillin+1% arabinose were removed from the refrigerator and warmed to room temperature. Multiple dilutions were used for each plating so as to have countable and isolated colonies on the plates. Plating suggestions:

TABLE 4

| | Dilution(s) | |
|---|---|---|
| Sequencing type | suggested | Volume to plate |
| SinglePlex | 10⁻¹ through 10⁻³ | 300 uL |
| Amplicon | None | 300 uL (= 1/10^{th} |
| | | recovery) |

After three hours, the culture tubes were removed from the shaking incubator. First, plating for amplicon sequencing was performed by following the above table. Plating beads were used to evenly distribute the culture over the agar. The beads were removed from the plate the plate was allowed to dry uncovered in a flow hood. While the plates were drying, the remaining culture was used to perform serial dilutions, where the standard dilutions were 50 μL of culture into 450 uL of sterile, 0.8% NaCl. The plate/tubes used for these dilutions (as well as the original culture) were maintained at 4° C. in case additional dilutions were needed to be performed based on colony counts. Plating for whole genome sequencing was performed according to the Table 4. Additional or fewer dilutions may be used based on library/competent cell knowledge. The cultures were evenly spread across the agar using sterile, plating beads. The beads were then removed from the plate and the plates were allowed to dry uncovered in the flow hood. While the plates were drying, an incubator was programmed according to the following settings: 30° C. for 9 hours→42° C. for 2 hours→30° C. for 9 hours. The agar plates were placed in the pre-set incubator, and after the temperature cycling was complete (˜21 hours), the agar plates were removed from the incubator. If induction of editing has been successful, size differences in the colonies will be visible.

### Example 6: Liquid Cell Culture Procedure for Comparison to Bulk Culture

Liquid Culture Process for Control:

The editing cassette libraries were transformed via electroporation into specific strains of E. coli expressing Mad7 (nuclease) and Lambda Red (recombination) proteins. Transformation of process control vectors—alongside the editing cassette libraries—is essential to calculate the transformation efficiency and editing efficiency (sgRNA efficiency). Immediately post-transformation, the electroporated cells were transferred to medium for recovery.

TABLE 5
Summary of Related QC Assays/M-Tools

| | QC Assay | What is being | | |
|---|---|---|---|---|
| Module | Description | measured | Input | Output |
| Transforma- | Live/dead | #starting | live/dead | # live/dead |
| tion | flow after | cells, # | stain, cells | cells, |
| (supercoiled | transforma- | live cells | after | selective/non- |
| plasmids) | tion | with plasmid | transf. | selective |
| | | | | conditions |
| Transforma- | Live/dead | #starting | live/dead | # live/dead |
| tion | flow after | cells, # | stain, cells | cells, |
| (direct to | transforma- | live cells | after | selective/non- |
| test) | tion | with plasmid | transf. | selective |
| | | | | conditions |

Following electroporation and recovery, cells from these process control transformations were spread on LB agar plates with the appropriate antibiotics. After overnight growth on plates, cells are scraped and then plated on the selective MacConkey phenotypic agar plates for the sugar edits that are tested: Xylose, Galactose, or Lactose, or scraped and replated on LB agar to determine clonality of the individual cells from plates. In more detail, after a 3-hour incubation (recovery), the culture tubes were removed from the shaking incubator. While the culture tubes were incubating, 250 mL baffled shake flasks were prepared with 25 mL of LB+100 ug/mL carbenicillin and 25 ug/mL chloramphenicol and 1% arabinose. After incubation, 250 μL of undiluted culture from each transformation was transferred into the prepared 250 mL shake flasks. An incubator was set to the following temperature settings: 30° C. for 9 hours→42° C. for 2 hours→30° C. for 9 hours. This temperature regime was used to allow for additional recovery during the first nine hours followed by induction of the nuclease during the two-hour step. The lambda (recombineering system) induction was triggered by arabinose in the medium. The flasks were incubated/shaken at 250 RPM. After the temperature cycling was complete (˜21 hours), the flasks were removed from the incubator/shaker.

Serial dilutions of each culture were prepared with 0.8% NaCl, where the standard dilutions were 50 μL of culture into 450 μL of sterile, 0.8% NaCl, and dilutions of 10⁻⁵ to 10⁻⁷ were made to produce isolated colonies. 300 uL of each dilution of each culture was plated onto LB agar plates with standard concentrations of chloramphenicol and carbenicillin. Arabinose was not used in the agar plates as all editing should have occurred in the incubation/shaking process. The plates were placed in a 30° C. incubator for overnight growth where colonies formed overnight and were picked for whole genome next-gen sequencing the following day using 250 μL of culture as the input for a plasmid extraction protocol.

### Example 7: Results

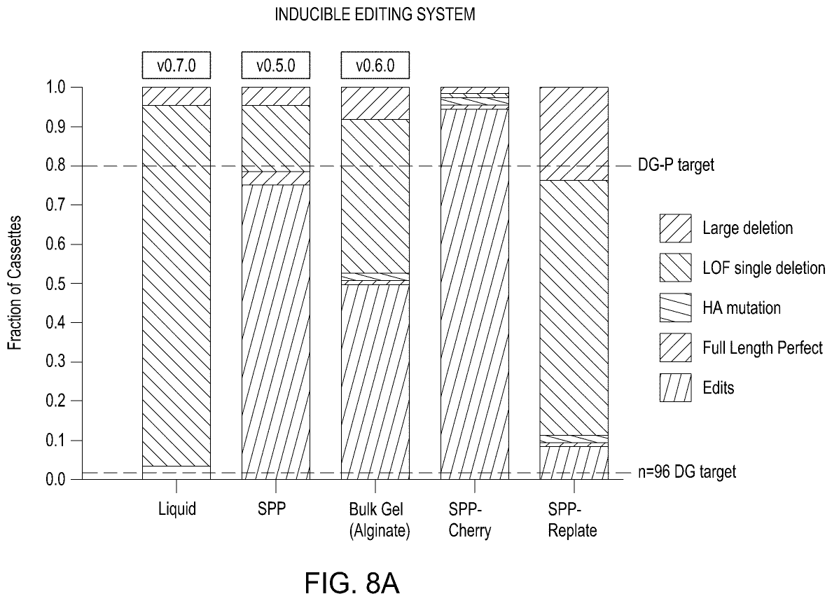

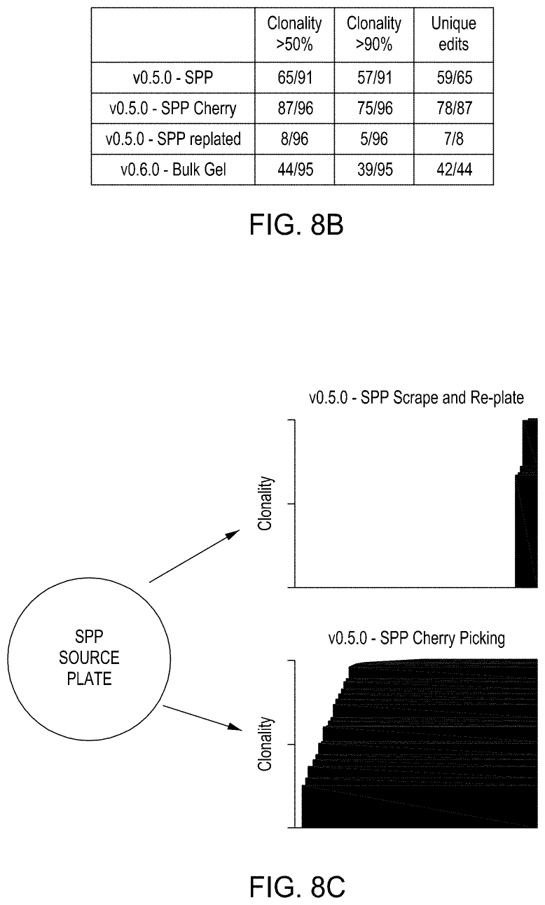

FIGS. 8A, 8B, and 8C show the results of the editing rates and clonality resulting from editing experiments performed with liquid cell culture employing no isolation or normalization, but employing inducible editing; bulk cell gel culture, employing isolation, inducible editing, and normalization; solid agar plating (SPP) employing isolation, inducible editing, and normalization; solid agar plating (SPP-Cherry) employing isolation, inducible editing, and cherry picking; and solid agar plating (SPP) employing only isolation and inducible editing and simply scraping the colonies from the plate and re-plating. In this context, “cherry” refers to cherry-picking of small colonies, which, when cells are plated and grown into colonies, it has been determined that small colonies are likely to be colonies of edited cells, where large, fast-growing colonies typically are non-edited cells (e.g., escapees). See, e.g., U.S. Pat. No. 10,253,316, filed 30 Jun. 2018; U.S. Pat. No. 10,329,559, filed 7 Feb. 2019; and U.S. Pat. No. 10,323,242, filed 7 Feb. 2019; and U.S. Ser. No. 16/412,175, filed 14 May 2019; Ser. No. 16/412,195, filed 14 May 2019; Ser. No. 16/454,865, filed 6 Jun. 2019; and Ser. No. 16/423,289, filed 28 May 2019, all of which are herein incorporated by reference in their entirety.

FIG. 8A shows that liquid culture results in a very low rate of observed editing, at about 1-2%; the standard plating procedure (SPP) results in an approximate 75% rate of observed editing in the cell population; the bulk alginate cell culture protocol results in an approximate 50% rate of observed editing; the standard plating procedure plus cherry picking (SPP-cherry) (e.g., manual picking of only small colonies from the plated cells, where the presumption is that small colonies represent colonies of cells that have been edited) protocol results in an approximate 95% rate of observed editing; and the standard plating procedure (SPP) without normalization or cherry picking results in an approximate 8% rate of observed editing. Thus, it is clear that SPP+cherry picking produces the highest rate of observed editing but requires manual intervention for picking colonies. In addition, SPP without cherry picking, but including isolation, induced editing, and normalization results in a high (75%) rate of observed editing, and the easily-automatable bulk gel cell culture process resulted in an approximate 50% rate of observed editing.

FIG. 8B provides the observed clonality for the standard plating procedure (SPP), the standard plating procedure+cherry picking (SPP cherry), the standard plating procedure+scraping the plate comprising the colonies where editing has been induced (but also comprising unedited cells), and for the bulk procedure. The first column gives the fraction of colonies examined with more than half the reads being called edits. The second column gives the fraction of colonies that have more than 90% of the reads being called edit reads. The higher fraction here shows how complete the edits are if there are some colonies examined between the 50% and 90% cut offs that demonstrate that not all of the cells in the colony that is being picked are edited. That is, when one cell hits the plate and begins growing into a clonal colony for, e.g., ˜100 cells—then editing is induced—some of the cells are edited but not all, and the cells that are not edited cause this incomplete editing in the colony (e.g., less than 100% clonality). The third column provides the number of unique edits for the colonies in the >50% clonal colonies. Note that SPP-cherry provides the highest clonality and number of unique edits, but that the bulk gel cell culture provides good clonality (44/95 at >50%) and a high proportion of the clonal colonies consist of unique edits (42/44).

Finally, FIG. 8C provides a graph of the data in FIG. 8B. This graph indicates the extent of incomplete editing.

### Example 8: Recursive Editing and Curing

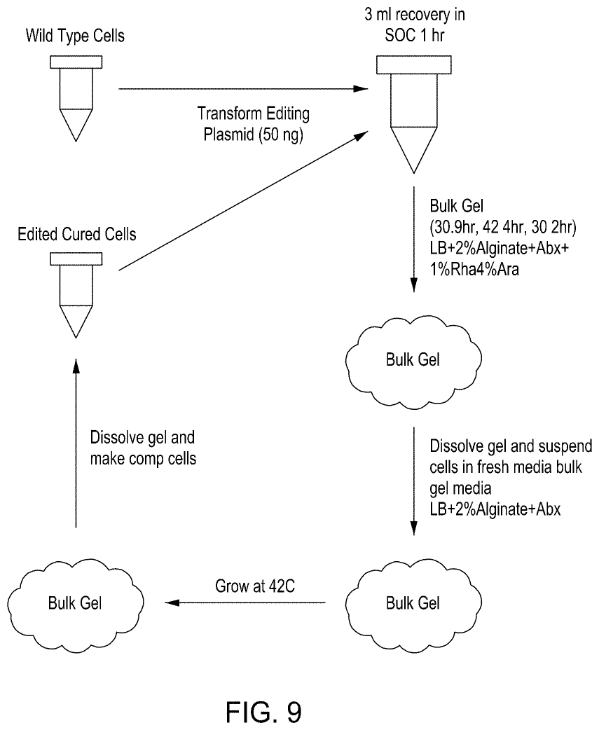

After the transformation, recovery, isolation, editing, and dissolving (as described above), cells were then pelleted (in a centrifuge or using a filter) and resuspended in fresh media. Cells were then grown in the bulk gel at 42° C., which induces curing of the plasmid (e.g., the combined engine/editing plasmid). Once cells have reached optimal growth phase the bulk gel was again dissolved and the cells were made competent by washing and concentrating in 10% glycerol using centrifugation or filtration). These cells were then transformed with a second combined engine/editing plasmid, and the isolation, growth, inducement of editing and normalization was performed as detailed above. The recursive process resulted in 33% of all cells with edits at both target sites. A recursive process is depicted in FIG. 9.

While this invention is satisfied by embodiments in many different forms, as described in detail in connection with preferred embodiments of the invention, it is understood that the present disclosure is to be considered as exemplary of the principles of the invention and is not intended to limit the invention to the specific embodiments illustrated and described herein. Numerous variations may be made by persons skilled in the art without departure from the spirit of the invention. The scope of the invention will be measured by the appended claims and their equivalents. The abstract and the title are not to be construed as limiting the scope of the present invention, as their purpose is to enable the appropriate authorities, as well as the general public, to quickly determine the general nature of the invention. In the claims that follow, unless the term “means” is used, none of the features or elements recited therein should be construed as means-plus-function limitations pursuant to 35 U.S.C. § 112, 916.

We claim:

1. A method for performing enrichment of cells edited by a nucleic acid-guided nuclease comprising:
providing transformed cells at a dilution resulting in isolated cells in an appropriate liquid growth medium comprising 0.5%-6% alginate, wherein the cells comprise one or more nucleic acid-guided nuclease editing components under the control of an inducible promoter;
solidifying the alginate-containing medium with a solution of a divalent cation;
providing conditions to allow nucleic acid-guided editing to proceed;
allowing the cell colonies to grow to become normalized; and
liquefying the alginate-containing medium with a solution comprising a divalent cation chelating agent.

2. The enrichment method of claim 1, wherein the nucleic acid-guided nuclease editing components are provided to the cells on a single vector.

3. The enrichment method of claim 1, wherein a coding sequence for a nuclease is provided on an engine vector and an editing cassette comprising a sequence for a gRNA and a donor DNA are provided on an editing vector.

4. The enrichment method of claim 1, wherein the cells are bacterial cells, yeast cells, or mammalian cells.

5. The enrichment method of claim 1, wherein the percentage of alginate in the growth medium is 1%-4%.

6. The enrichment method of claim 4, wherein the percentage of alginate in the growth medium is 2%-3%.

7. The enrichment method of claim 1, wherein editing is induced and transcription of the gRNA is under the control of an inducible promoter.

8. The enrichment method of claim 7, wherein the inducible promoter is a pL promoter.

9. The enrichment method of claim 7, wherein transcription is induced by raising temperature of the cells to 42° C.

10. The enrichment method of claim 7, wherein, before induction of transcription of the gRNA, the isolated cells grow for 2-50 doublings at 30° C. for 6-12 hours.

11. An automated multi-module cell processing instrument for performing automated enrichment of cells edited by a nucleic acid-guided nuclease editing comprising:
a cell receptacle configured to hold cells;
a nucleic acid receptacle configured to hold editing vectors;
a reagent cartridge configured to hold reagents;
a growth module;
a filtration module;
a transformation module;
a dilution module;
an isolation module comprising a temperature controlled vessel configured to perform the solidifying, allowing, providing, allowing and liquefying steps of claim 1; and
a liquid handling system configured to transfer liquids from the cell receptacle to the growth module, from the growth module to the filtration module, from the filtration module to the transformation module, from the nucleic acid receptacle to the transformation module, from the transformation module to the dilution module and from the dilution module to the isolation module without user intervention.

12. The instrument for performing automated enrichment of cells according to claim 11, wherein the gRNA is under transcriptional control of an inducible promoter and the inducible promoter is a temperature inducible promoter.

13. The instrument for performing automated enrichment of cells according to claim 11, wherein the reagent cartridge comprises medium, a solution of a divalent cation, and a solution for a chelating agent of a divalent cation.

14. The automated multi-module cell processing instrument of claim 13, wherein the divalent cation is CaCl₂ and the chelating agent is Na₃C₆H₅O₇.

15. The automated multi-module cell processing instrument of claim 11, wherein the transformation module comprises a flow-through electroporation device.

16. The automated multi-module cell processing instrument of claim 11, wherein the filtration module comprises a tangential flow filtration device.

17. A method for performing enrichment of cells edited by a nucleic acid-guided nuclease comprising:
providing transformed cells at a dilution in a vessel resulting in isolated cells in an appropriate liquid growth medium comprising a hydrogel, wherein the cells comprise nucleic acid-guided nuclease editing components under the control of an inducible promoter;
solidifying the hydrogel-containing medium with a solution of a divalent cation;
allowing the isolated cells to grow for 2 and 50 doublings to establish cell colonies;
inducing transcription of the nucleic acid guided-nuclease editing components;
growing the cells for a period of time sufficient to allow the cell colonies to become normalized; and
liquefying the hydrogel-containing medium with a chelating agent for the divalent cation.
